(19) United States
(12) Patent Application Publication
(10) Pub. No.: US 2025/0322916 A1
(43) Pub. Date: Oct. 16, 2025

(54) ADAPTIVE DISCOVERY AND MIXED-VARIABLE OPTIMIZATION OF NEXT GENERATION SYNTHESIZABLE MICROELECTRONIC MATERIALS

(71) Applicants: NORTHWESTERN UNIVERSITY, Evanston, IL (US); MASSACHUSETTS INSTITUTE OF TECHNOLOGY, Cambridge, MA (US)

(72) Inventors: Wei Chen, Wilmette, IL (US); James Michael Rondinelli, Evanston, IL (US); Ramin Baghgar Bostanabad, Irvine, CA (US); Elsa Olivetti, Cambridge, MA (US); Daniel Apley, Evanston, IL (US)

(21) Appl. No.: 18/569,628

(22) PCT Filed: Jun. 16, 2022
(86) PCT No.: PCT/US2022/033892

§ 371 Date: Dec. 13, 2023

Related U.S. Application Data
(60) Provisional application No. 63/211,603, filed on Jun. 17, 2021.

(51) Int. Cl. G16C 20/50; G16C 20/64; G16C 20/70

(52) U.S. Cl. CPC G16C 20/50; G16C 20/64; G16C 20/70

(57) ABSTRACT

This invention relates to systems and methods for adaptive discovery and mixed-variable optimization of synthesizable microelectronic materials, and applications of the same. Specifically, an exemplary system includes a virtual screening (VS) module to extract information from literatures of a knowledge base by text mining, a ML-assisted conceptual exploration (CE) module to identify candidate material families for the specific class of compound materials based on the extracted information via a combination of ML models and to generate exogenous models of objective functions f(x, y) and constraint functions g(x, y), and an adaptive discovery (AD) engine to generate and optimize design of the newly discovered compound materials. The AD engine includes a mixed-variable ML module, a mixed-integer optimization (MIO) module, and a high-fidelity evaluation (HFE) module, which are iteratively and sequentially executed.

### CROSS-REFERENCE TO RELATED APPLICATIONS

This PCT application claims priority to and the benefit of U.S. Provisional Patent Application Ser. No. 63/211,603, which was filed Jun. 17, 2021. The content of the application is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### FIELD OF THE INVENTION

The present invention relates generally to hypothesis generation (conceptual design) of materials/molecules, and more particularly to systems and methods for adaptive discovery and mixed-variable optimization of synthesizable microelectronic materials, and applications of the same.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Hypothesis generation (conceptual design) of new materials is characterized by several challenges such as high-dimensionality of the atomic structure-composition variable space, formidable cost of directly using high-fidelity simulations for design optimization, dispersity in literature-reported similar materials and synthesis methods, complex physical mechanisms, and mixed qualitative and quantitative design variables that lead to a disjointed design space. Even though machine learning (ML) techniques have been employed to expedite materials innovation, existing methods treat ML and design optimization as two separate processes, failing to resolve the fundamental challenges associated with high dimensionality and mixed-variable complexity.

Therefore, a heretofore unaddressed need exists in the art to address the aforementioned deficiencies and inadequacies.

### SUMMARY OF THE INVENTION

Certain aspects of the invention relate to systems and methods for adaptive discovery and mixed-variable optimization of next generation synthesizable microelectronic materials, and applications of the same.

In one aspect, the invention relates to a system for performing machine learning (ML) enhanced conceptual design of compound materials. In certain embodiments, the system includes a computing device comprising at least one processor and a storage device storing computer executable code. The computer executable code, when executed at the at least one processor, includes: a data repository, configured to store, for a specific class of compound materials, information of existing and newly discovered compounds; a virtual screening (VS) module, configured to identify and obtain, from literatures of a knowledge base, extracted information related to key material descriptors, relevant materials, and associated synthesis procedures of the specific class of compound materials; a ML-assisted conceptual exploration (CE) module, configured to identify candidate material families for the specific class of compound materials based on the extracted information via a combination of ML models, and to generate exogenous models of objective functions f(x, y) and constraint functions g(x, y), wherein x represents quantitative variables and y represents qualitative variables related to structures and synthesis parameters of the specific class of compound materials; and an adaptive discovery (AD) engine, configured to generate and optimize design of the newly discovered compound materials, and to add the information of the newly discovered compound materials to the data repository.

Specifically, the AD engine includes: a mixed-variable ML module, configured to perform mixed variable ML on the objective functions f(x, y) and constraint functions g(x, y) using a latent variable Gaussian process (LVGP) model; a mixed-integer optimization (MIO) module, configured to select new samples of (x, y) combinations in Bayesian optimization (BO) using the LVGP model and the objective functions f(x, y) and constraint functions g(x, y) for mixed-integer nonlinear programming (MINLP); and a high-fidelity evaluation (HFE) module, configured to perform density functional theory (DFT) simulation based on the candidate material families and the associated synthesis procedures. The HFE module, the mixed-variable ML module and the MIO module of the AD engine are iteratively and sequentially executed.

In another aspect, a method for performing ML enhanced conceptual design of compound materials includes: providing a knowledge base with literatures related to a specific class of compound materials; performing virtual screening (VS) using a VS module to identify and obtain, from the literatures of the knowledge base, extracted information related to key material descriptors, relevant materials, and associated synthesis procedures of the specific class of compound materials; performing ML-assisted conceptual exploration (CE) using a CE module to identify candidate material families for the specific class of compound materials based on the extracted information via a combination of ML models, and to generate exogenous models of objective functions f(x, y) and constraint functions g(x, y), wherein x represents quantitative variables and y represents qualitative variables related to structures and synthesis parameters of the specific class of compound materials; and performing adaptive discovery (AD) using an AD engine to generate and optimize design of the newly discovered compound materials, and to add the information of the newly discovered compound materials to a data repository. The AD engine includes: a mixed-variable ML module, configured to perform mixed variable ML on the objective functions f(x, y) and constraint functions g(x, y) using a latent variable Gaussian process (LVGP) model; a mixed-integer optimization (MIO) module, configured to select new samples of (x, y) combinations in Bayesian optimization (BO) using the LVGP model and the objective functions f(x, y) and constraint functions g(x, y) for mixed-integer nonlinear programming (MINLP); and a high-fidelity evaluation (HFE) module, configured to perform density functional theory (DFT) simulation based on the candidate material families and the associated synthesis procedures. The HFE module, the mixed-variable ML module and the MIO module of the AD engine are iteratively and sequentially executed.

In certain embodiments, the VS module is a natural language processing (NLP) based VS module, comprising: a data retrieval module configured to download the literatures using an Application Programming Interface (API) and retrieve content texts from the literatures; and a text mining module, configured to perform text mining on the content texts using an unsupervised probabilistic model to obtain the extracted information.

In certain embodiments, the literatures include journal articles and patents.

In certain embodiments, the text mining module comprises: a paragraph classifier configured to performing paragraph classifying on the content texts to identify paragraphs of interest; a token classifier configured to tokenize words of interest within the paragraphs of interest and label the tokenized words to identify the relevant materials as recognized entities; and a recipe mapper module, configured perform entity linking to map the recognized entities to relevant information of the knowledge base, and to establish connections between entities.

In certain embodiments, the ML models of the ML-assisted CE module comprise design of experiments (DoE) based active learning, nonlinear regression and classification, and conditional variational autoencoders (CVAEs).

In certain embodiments, the specific class of compound materials is a metal-insulation transitions (MITs) compounds.

In certain embodiments, the relevant materials includes: known MITs compounds; unidentified potential MITs compounds with shared similarities; and non-MITs materials.

In certain embodiments, the ML-assisted CE module is configured to: receive the extracted information from the VS module as input; perform initial DoE and CVAE deep learning feature extraction to capture all known MITs and non-MITs compounds of the specific class of compound materials for subsequent model training and validation; construct a classification model using existing dataset of compositions and structures of MITs and relevant non-MITs compounds extracted, raw candidate MIT materials and possible synthesis parameters, latent space representation of existing MITs materials, frequently used keywords in existing papers on MITs materials, all existing MITs materials, and relevant non-MITs materials, and predict, using the classification model, the candidate material families; perform active learning of responses with regression models; perform CVAE deep learning for generating synthesis recipes; and obtain exogenous regression models for cost and performance.

In certain embodiments, in the mixed variable ML module, the LVGP model performs latent variable mapping to transform the qualitative variables y into latent variables z in a two dimensional (2D) latent space to achieve physics-based dimension reduction.

In certain embodiments, the quantitative variables x comprise: operating pressure of a material; stress of the material; temperature of the material; carrier density of the material; fractional site occupancy of the material; synthesis time; synthesis temperature; synthesis pressure; and synthesis pH value.

In certain embodiments, the qualitative variables y comprise: architecture of a material; stoichiometry of the material; composition of the material; type of reaction; and processing procedure.

In yet another aspect of the invention, a non-transitory tangible computer-readable medium is provided for storing instructions which, when executed by one or more processors, cause the method as discussed above to be performed.

These and other aspects of the invention will become apparent from the following description of the preferred embodiment taken in conjunction with the following drawings, although variations and modifications therein may be affected without departing from the spirit and scope of the novel concepts of the invention.

### BRIEF DESCRIPTION OF THE DRAWINGS

The following drawings form part of the present specification and are included to further demonstrate certain aspects of the invention. The invention may be better understood by reference to one or more of these drawings in combination with the detailed description of specific embodiments presented herein. The drawings described below are for illustration purposes only.

The drawings are not intended to limit the scope of the present teachings in any way.

FIG. 1A schematically shows a system according to certain embodiments of the present invention.

FIG. 1B schematically shows the virtual screening (VS) module of the system as shown in FIG. 1A according to certain embodiments of the present invention.

FIG. 1C schematically shows a functional block diagram of a system according to certain embodiments of the present invention.

FIG. 2 shows resistivity and transition temperature ranges for many known metal-insulation transitions (MITs) materials according to certain embodiments of the present invention.

FIG. 3 shows design space for large MITs materials according to certain embodiments of the present invention.

FIG. 4 shows a table of performance comparison between existing technologies and the proposed approach for the system according to certain embodiments of the present invention.

FIG. 5 shows a table of a preliminary list of words and phrases to create embeddings in text minding according to certain embodiments of the present invention.

FIG. 6 schematically shows text extraction workflow of the VS module according to certain embodiments of the present invention.

FIG. 7 shows ML-assisted concept exploration according to certain embodiments of the present invention, where (a) shows domain-science understanding of relation between local structural distortions and physical interactions in MITs compounds; (b) shows SHAP feature importance from Rondinelli's MITs classification model with colorcoded relative feature value.

The horizontal location, SHAP value, quantifies feature impact on MITs prediction. (c) shows cluster analysis by the top two features separating MITs and non-MITs materials.

FIG. 8 shows latent representation in a LVGP model according to certain embodiments of the present invention.

FIG. 9 shows the LVGP model enabling Bayesian optimization for mixed-integer variables according to certain embodiments of the present invention.

FIG. 10 shows the band gap (a) optimization history, (b) evaluation against the BO prediction and DFT calculations, (c) model representation in latent space for the B site in the lacunar spinels according to certain embodiments of the present invention.

FIG. 11 shows a table of examples of MITs design variables, constraints and objectives according to certain embodiments of the present invention.

FIG. 12 shows a table of representative recipe paragraphs and relevant synthesis information extracted from the recipe paragraphs according to certain embodiments of the present invention, including the classified synthesis method, target(s) and precursor(s), and other relevant synthesis information including temperatures and time.

FIG. 13 shows representative violin plots of extracted temperatures of heat treatment processes in sol-gel and solid-state reaction synthesis methods according to certain embodiments of the present invention.

FIG. 14 shows Classification of lacunar spinels predicted by the multi-objective LVGP based on the online MIT classifier pipeline according to certain embodiments of the present invention.

FIG. 15 shows a phase diagram with TMIT for the RNiO3 perovskite family versus Average Deviation of the Covalent Radius. LaNiO3 is always metallic, whereas the other nickelates exhibit MITs according to certain embodiments of the present invention.

FIG. 16 shows SHAP force plot of the T classifier's prediction for the MIT compounds LuNiO₃ and NdNiO₃ according to certain embodiments of the present invention.

FIG. 17 shows interplay of ADCR and Global Instability Index according to certain embodiments of the present invention.

FIG. 18 shows a table of current ongoing tasks regarding CVAE model preparation and deployment according to certain embodiments of the present invention.

FIG. 19 shows interplay of ADCR and Ewald Energy per Atom, where for binary materials families, VₙOₘ and TiOₘ, the Ewald energy is a strong predictor of metallic, MIT, or insulating behavior, with the most ionic (lowest Ewald energy) presenting insulating behavior, most covalent showing metallic behavior, and MIT behavior in between.

FIG. 20 shows (a) an example of a variational autoencoder architecture, and (b) Depiction of the joint CVAE model architecture.

FIG. 21 shows sample predictions generated by the precursor CVAE for query target materials.

FIG. 22 shows interplay of the distance between identical transition metal ions and the unscreened Hubbard U interaction, where the ALE plot (left) shows the contribution to the classification probability from these two features, with a higher value (red) corresponding to a higher probability of a positive MIT classification; and the scatter plot (right) shows the distribution of compounds in the dataset as a function of these two features, with select families labeled.

FIG. 23 shows subset of compounds identified by high-throughput screening of Materials Project with the MIT classification model and their probabilistic class labels.

FIG. 24 shows overview of precursor CVAE model with anionic conditioning considered.

In the example, the shared target element is La and the target anionic element is 0.

FIG. 25 shows DFT-simulated electronic properties of selected lacunar spinel compositions at the Pareto front according to certain embodiments of the present invention.

FIG. 26 shows an example of the coupled electronic and structural mechanism in the MIT transition of Ca₂RuO₄ as captured by the calculations according to certain embodiments of the present invention.

FIG. 27 shows (left) schematic of a unit cell of a RP compound. (right) illustration of different magnetic states the inventors consider in the Ruddlesden-Popper perovskite optimization.

FIG. 28 shows (left) Illustration of the Ca₂RuO₄ electronic configuration in the insulating state, where the 4 electrons and 2 holes break symmetry in the 3-orbital t2g manifold, and the Ca₂MoO₄ electronic configuration (center) is electron-hole symmetric; and (right) suggested pathways to synthesize Ca₂MoO₄ from the Olivetti group.

FIG. 29 shows DFT-evaluated ground state properties of the Pareto-front compounds for the spinel materials family. NOI stands for number of MOBO iterations needed to discover them. ⅓ of the compounds are unstable, however they also have the largest band gap. Values of ΔHd (in units of eV/formula unit) that are positive indicate that the compound is stable. Eg is the DFT band gap in eV FIG. 30 shows illustration of the property requirements for a RP compound to be identified as a viable MIT material.

FIG. 31 shows a design of experiment for complex lacunar spinel family according to certain embodiments of the present invention.

FIG. 32 shows (a) a direct approach and (b) a proposed approach of the LVGP-SVI model according to certain embodiments of the present invention.

FIG. 33 shows a table of replicated 10-fold CV estimates of the relative root mean-squared error (RRMSE) for different models and the time to train the final model on the entire dataset according to certain embodiments of the present invention.

FIG. 34 illustrates LVGP-SI with logistic projections.

FIG. 35 shows a table of replicated 10-fold CV estimates of the relative root mean-squared error (RRMSE) for the different LVGP-SVI optimization approaches.

FIG. 36 shows boxplots of the MSEs of each model, across different CV splits: (a) on the whole (270) dataset and on subsets of size (b) 100, (c) 50, and (d) 20. “s” and “m” refer to “SR-LVGP” and “MR-LVGP” respectively.

FIG. 37 shows CV r² of the models in the test cases. The n=20 cases are not shown, for r² goes below 0 due to lack of sufficient training data.

FIG. 38 shows boxplots of the MSEs of each model, across different CV splits; (a) on the complete (270) dataset, (b) on the subset (50) data.

FIG. 39 shows (a) Scatter plot of stability-formation energy of the ternary oxide dataset. (b) Scatter plot of stability-bandgap of the MITs dataset. Values of each variable are rescaled to [0,1].

FIG. 40 shows performance of BO on the M2AX dataset with Bayesian LVGP as the surrogate model using weakly informative and informative priors.

FIG. 41 shows representations for qualitative variables in (a) Cartesian, (b) hyperspherical, and (c) mixed latent space in 2-dimensional (d=2) case.

FIG. 42 shows results from multi-objective Bayesian optimization of lacunar spinels according to certain embodiments of the present invention, where (a) shows the maximum value of EMI and cumulative percentage of Pareto Front compositions identified within 60 iterations. Red asterisk marks iterations when a Pareto front composition is identified, (b) shows optimization history in objective space, (c) shows consolidated result from 10 trials of MOBO, each time initialized with a different DoE, (d) and (e) show consolidated result from 10 trials of single objective Bayesian optimization for bandgap (d) and stability (e).

FIG. 43 shows comparison of best design identified by Bayesian Optimization (BO) and Genetic Algorithm (GA) after 25 objective function evaluations of 2D Branin function (left) and 3D Hartmann function (right).

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this specification will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks.

The use of highlighting has no influence on the scope and meaning of a term; the scope and meaning of a term are the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a” “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures. is turned over, elements described as being on the “lower” side of other elements would then be oriented on “upper” sides of the other elements. The exemplary term “lower”, can, therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this specification, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and this specification, and will not be interpreted in an idealized or overly formal sense unless expressly so defined herein.

As used in this specification, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in this specification, the phrase “at least one of A, B, and C” should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in a different order (or concurrently) without altering the principles of the invention.

### OVERVIEW OF THE INVENTION

As discussed above, even though ML techniques have been employed to expedite materials innovation, existing methods treat ML and design optimization as two separate processes, failing to resolve the fundamental challenges associated with high dimensionality and mixed-variable complexity.

In response, the inventors propose an ML enhanced mixed-variable conceptual design optimization framework to efficiently extract useful information from existing data in literature and physics-based simulations to guide the autonomous search for optimal materials. Integrating five computational modules in an iterative process, the novelty lies in two main aspects: (1) Creativity: Integrated natural language processing (NLP) and physics-based ML for Virtual Screening, Concept Exploration, and Adaptive Discovery of candidate materials architectures, with co-design consideration of synthesis feasibility and iterations with subsequent ML-based optimization; and (2) Efficiency: A novel latent-variable Gaussian process (LVGP) ML approach for mixed-variable problems with uncertainty quantification, which seamlessly integrates with Bayesian reinforcement learning and optimization and achieves superb efficiency through embedded physics-based dimension reduction.

One of the objectives of the invention is to develop and employ ML enhanced mixed-variable design optimization for accelerated hypothesis generation (conceptual design) of new functional materials in energy innovations. The project is motivated by the challenges in developing intelligent computational algorithms to accelerate the discovery and design of new materials with enormous atomic structure-composition variable spaces (>106 of design options) and computational cost (>hours or days for each evaluation), unknown synthesis feasibility, complex physical mechanisms varying among different architectures, and a mixture of qualitative and quantitative design variables covering materials structure and synthesis parameters. The computational ML and design framework will rapidly explore materials architectures and optimize their compositions by bridging the gap between the knowledge in literature and that discovered from physics-based simulations. Through iterative adaptive discovery, rare-event discoveries (every few years for a new material) will be transformed to persistent innovations.

In one aspect, the invention relates to a system for performing machine learning (ML) enhanced conceptual design of compound materials. In certain embodiments, the system includes a computing device comprising at least one processor and a storage device storing computer executable code. The computer executable code, when executed at the at least one processor, includes software modules comprising: a data repository, configured to store, for a specific class of compound materials, information of existing and newly discovered compounds; a virtual screening (VS) module, configured to identify and obtain, from literatures of a knowledge base, extracted information related to key material descriptors, relevant materials, and associated synthesis procedures of the specific class of compound materials; a ML-assisted conceptual exploration (CE) module, configured to identify candidate material families for the specific class of compound materials based on the extracted information via a combination of ML models, and to generate exogenous models of objective functions f(x, y) and constraint functions g(x, y), wherein x represents quantitative variables and y represents qualitative variables related to structures and synthesis parameters of the specific class of compound materials; and an adaptive discovery (AD) engine, configured to generate and optimize design of the newly discovered compound materials, and to add the information of the newly discovered compound materials to the data repository. Specifically, the AD engine includes: a mixed-variable ML module, configured to perform mixed variable ML on the objective functions f(x, y) and constraint functions g(x, y) using a latent variable Gaussian process (LVGP) model, wherein the LVGP model performs latent variable mapping to transform the qualitative variables y into latent variables z in a two dimensional (2D) latent space to achieve physics-based dimension reduction; a mixed-integer optimization (MIO) module, configured to select new samples of (x, y) combinations in Bayesian optimization (BO) using the LVGP model and the objective functions f(x, y) and constraint functions g(x, y) for mixed-integer nonlinear programming (MINLP); and a high-fidelity evaluation (HFE) module, configured to perform density functional theory (DFT) simulation based on the candidate material families and the associated synthesis procedures. The HFE module, the mixed-variable ML module and the MIO module of the AD engine are iteratively and sequentially executed.

In another aspect, a method for performing ML enhanced conceptual design of compound materials includes: providing a knowledge base with literatures related to a specific class of compound materials; performing virtual screening (VS) using a VS module to identify and obtain, from the literatures of the knowledge base, extracted information related to key material descriptors, relevant materials, and associated synthesis procedures of the specific class of compound materials; performing ML-assisted conceptual exploration (CE) using a CE module to identify candidate material families for the specific class of compound materials based on the extracted information via a combination of ML models, and to generate exogenous models of objective functions f(x, y) and constraint functions g(x, y), wherein x represents quantitative variables and y represents qualitative variables related to structures and synthesis parameters of the specific class of compound materials; and performing adaptive discovery (AD) using an AD engine to generate and optimize design of the newly discovered compound materials, and to add the information of the newly discovered compound materials to a data repository. The AD engine includes: a mixed-variable ML module, configured to perform mixed variable ML on the objective functions f(x, y) and constraint functions g(x, y) using a latent variable Gaussian process (LVGP) model; a mixed-integer optimization (MIO) module, configured to select new samples of (x, y) combinations in Bayesian optimization (BO) using the LVGP model and the objective functions f(x, y) and constraint functions g(x, y) for mixed-integer nonlinear programming (MINLP); and a high-fidelity evaluation (HFE) module, configured to perform density functional theory (DFT) simulation based on the candidate material families and the associated synthesis procedures. The HFE module, the mixed-variable ML module and the MIO module of the AD engine are iteratively and sequentially executed.

In yet another aspect of the invention, a non-transitory tangible computer-readable medium is provided for storing instructions which, when executed by one or more processors, cause the method as discussed above to be performed.

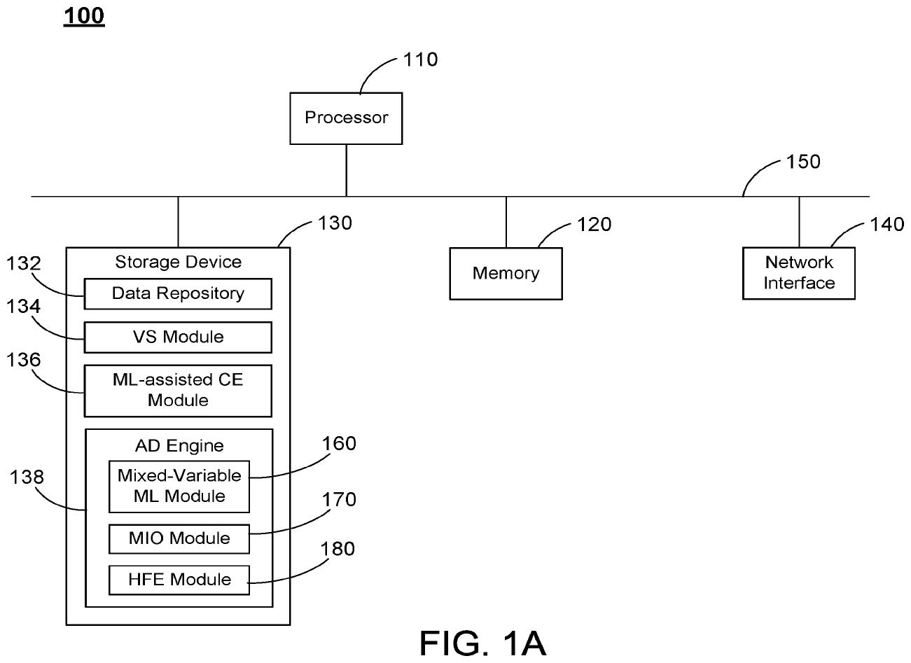

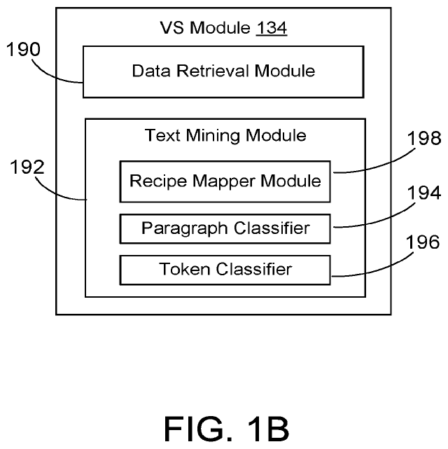

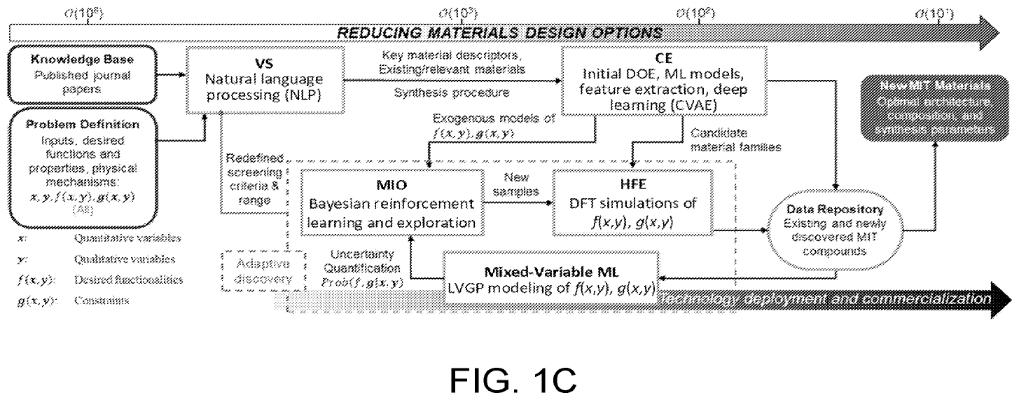

In certain embodiments, the system for adaptive discovery and mixed-variable optimization of synthesizable microelectronic materials can be implemented with an ML-enhanced conceptual design framework that connects five computational modules. For example, FIG. 1A schematically shows a system according to certain embodiments of the present invention. Specifically, the system 100 as shown in FIG. 1A is in the form of a computing device. As shown in FIG. 1A, the computing device 100 includes a processor 110, a memory 120, a storage device 130, a network interface 140, and a bus 150 interconnecting the processor 110, the memory 120, the storage device 130 and the network interface 140. In certain embodiments, the computing device 100 may include necessary hardware and/or software components (not shown) to perform its corresponding tasks. Examples of these hardware and/or software components may include, but not limited to, other required memory modules, interfaces, buses, Input/Output (I/O) modules and peripheral devices, and details thereof are not elaborated herein.

The processor 110 controls operation of the computing device 100, which may be used to execute any computer executable code or instructions. In certain embodiments, the processor 110 may be a central processing unit (CPU), and the computer executable code or instructions being executed by the processor 110 may include an operating system (OS) and other applications, codes or instructions stored in the computing device 100. In certain embodiments, the computing device 100 may run on multiple processors, which may include any suitable number of processors.

The memory 120 may be a volatile memory module, such as the random-access memory (RAM), for storing the data and information during the operation of the computing device 100. In certain embodiments, the memory 120 may be in the form of a volatile memory array. In certain embodiments, the computing device 100 may run on more than one memory 120.

The network interface 140 is an interface for communication with the network. In certain embodiments, the network interface 140 may be an Ethernet interface.

The storage device 130 is a non-volatile storage media or device for storing the computer executable code or instructions, such as the OS and the software applications for the computing device 100. Examples of the storage device 130 may include flash memory, memory cards, USB drives, or other types of non-volatile storage devices such as hard drives, floppy disks, optical drives, or any other types of data storage devices. In certain embodiments, the computing device 100 may have more than one storage device 130, and the software applications of the computing device 100 may be stored in the more than one storage device 130 separately.

As shown in FIG. 1A, the computer executable code stored in the storage device 130 may include a data repository 132, a virtual screening (VS) module 134, a ML-assisted conceptual exploration (CE) module 136 and an adaptive discovery (AD) engine 138. The data repository 132 is a data store for storing, for a specific class of compound materials, information of existing and newly discovered compounds. The VS module 134 is a software module which, when executed, is used to identify and obtain, from literatures of a knowledge base, extracted information related to key material descriptors, relevant materials, and associated synthesis procedures of the specific class of compound materials. The ML-assisted CE module 136 is a software module which, when executed, is used to identify candidate material families for the specific class of compound materials based on the extracted information via a combination of ML models, and to generate exogenous models of objective functions f(x, y) and constraint functions g(x, y). Specifically, x represents quantitative variables and y represents qualitative variables related to structures and synthesis parameters of the specific class of compound materials. The AD engine 138 is a multi-module engine which is used to generate and optimize design of the newly discovered compound materials, and to add the information of the newly discovered compound materials to the data repository.

In certain embodiments, the VS module 134 may be a natural language processing (NLP) based VS module. For example, FIG. 1B schematically shows the virtual screening (VS) module of the system as shown in FIG. 1A according to certain embodiments of the present invention. As shown in FIG. 1B, the VS module 134 includes: a data retrieval module 190 configured to download the literatures using an Application Programming Interface (API) and retrieve content texts from the literatures; and a text mining module 192 configured to perform text mining on the content texts using an unsupervised probabilistic model to obtain the extracted information. In certain embodiments, the literatures include journal articles and patents. Further, the text mining module 192 includes: a paragraph classifier 194 configured to performing paragraph classifying on the content texts to identify paragraphs of interest; a token classifier 196 configured to tokenize words of interest within the paragraphs of interest and label the tokenized words to identify the relevant materials as recognized entities; and a recipe mapper module 198 configured perform entity linking to map the recognized entities to relevant information of the knowledge base, and to establish connections between entities.

In certain embodiments, the ML models of the ML-assisted CE module comprise design of experiments (DoE) based active learning, nonlinear regression and classification, and conditional variational autoencoders (CVAEs). In one embodiment, the ML-assisted CE module 136 is configured to: receive the extracted information from the VS module as input; perform initial DoE and CVAE deep learning feature extraction to capture all known MITs and non-MITs compounds of the specific class of compound materials for subsequent model training and validation; construct a classification model using existing dataset of compositions and structures of MITs and relevant non-MITs compounds extracted, raw candidate MIT materials and possible synthesis parameters, latent space representation of existing MITs materials, frequently used keywords in existing papers on MITs materials, all existing MITs materials, and relevant non-MITs materials, and predict, using the classification model, the candidate material families; perform active learning of responses with regression models; perform CVAE deep learning for generating synthesis recipes; and obtain exogenous regression models for cost and performance.

Referring back to FIG. 1A, the AD engine 138 includes: a mixed-variable ML module 160, configured to perform mixed variable ML on the objective functions f(x, y) and constraint functions g(x, y) using a LVGP model, where the LVGP model performs latent variable mapping to transform the qualitative variables y into latent variables z in a two dimensional (2D) latent space to achieve physics-based dimension reduction; a mixed-integer optimization (MIO) module 170, configured to select new samples of (x, y) combinations in Bayesian optimization (BO) using the LVGP model and the objective functions f(x, y) and constraint functions g(x, y) for mixed-integer nonlinear programming (MINLP); and a high-fidelity evaluation (HFE) module 180, configured to perform density functional theory (DFT) simulation based on the candidate material families and the associated synthesis procedures. The HFE module 180, the mixed-variable ML module 160 and the MIO module 170 of the AD engine 138 are iteratively and sequentially executed.

In certain embodiments, in the mixed variable ML module, the LVGP model performs latent variable mapping to transform the qualitative variables y into latent variables z in a two dimensional (2D) latent space to achieve physics-based dimension reduction.

In certain embodiments, the quantitative variables x comprise: operating pressure of a material; stress of the material; temperature of the material; carrier density of the material; fractional site occupancy of the material; synthesis time; synthesis temperature; synthesis pressure; and synthesis pH value. In certain embodiments, the qualitative variables y comprise: architecture of a material; stoichiometry of the material; composition of the material; type of reaction; and processing procedure.

FIG. 1C schematically shows a functional block diagram of a system according to certain embodiments of the present invention. As shown in FIG. 1C, the system includes the VS module, the ML-assisted CE module and the AD engine, where the AD engine is formed by the HFE module, the mixed-variable ML module and the MIO module that are iteratively and sequentially executed. In particular, the execution of the framework starts from Problem Definition and gathering the Knowledge Base (published journal papers) as inputs to Natural Language Processing (NLP)-based VS to identify relevant materials, associated synthesis procedures, and key materials descriptors by text mining the literature. The extracted information will then be used in CE to identify candidate material families via a combination of ML techniques: design of experiments (DoE) based active learning, nonlinear regression and classification, and conditional variational autoencoders (CVAEs). The generative aspect of the custom-architecture CVAE will create new candidate families and synthesis procedures for DFT based HFE. Next, the Mixed-Variable ML module will build a probabilistic surrogate on the obtained data using the LVGP method that transforms qualitative variables y into latent variables z in a supervised-learning manner, while embodying their effects on f(xy) and g(x,y). In MIO, using the LVGP model and other exogenous f and g functions passed from CE, a reinforcement learning strategy that balances exploration and exploitation, will select new samples in BO. The iteration adds new material designs to a Data Repository where the optimal architecture, composition, and synthesis parameters will be identified for technology transfer.

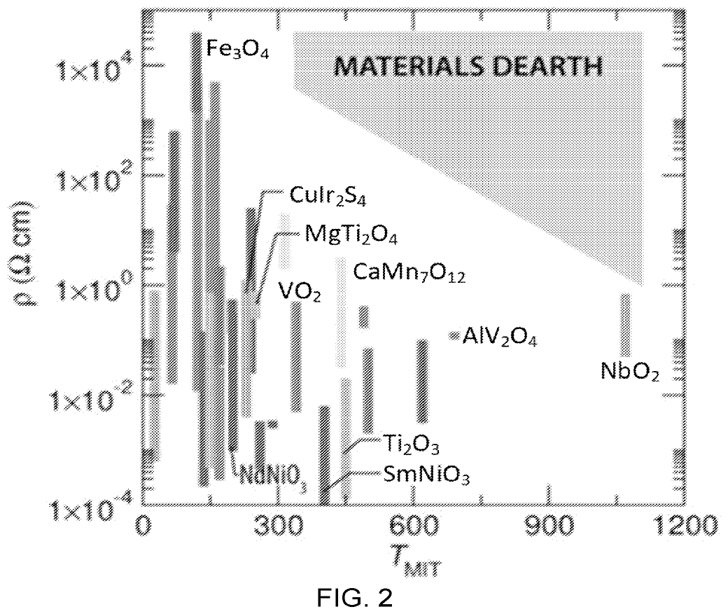

In certain embodiments, the specific class of compound materials exhibit a metal-insulator transition (MIT) or equivalently an insulator-metal transition (IMT). Specifically, the design of functional materials exhibiting MITs may be used as a testbed to demonstrate the effectiveness of the approach of the system. MITs compounds are a class of quantum materials that can revolutionize microelectronics science and technology by providing energy saving solutions. The ability to control sufficient charge densities within structures of a few-atom dimensions has been a major limitation of silicon-based microelectronic devices that rely on conventional transistor technologies. None of the known MITs materials (as shown in FIG. 2) achieve the reversible resistivity changes (˜10⁵) near room temperature required to outperform silicon.

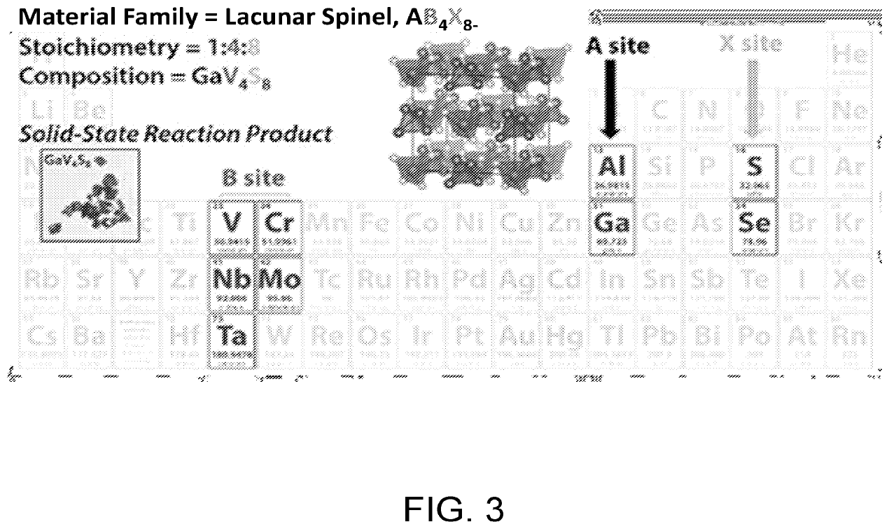

There are some major challenges and limitations of current technologies and design approaches. For example, considerable variations in material family (architecture), stoichiometry, and composition have resulted in a large design space of atomic structure-composition (>10⁶ options) of prospective MITs materials. Here, families are defined as structure prototypes with unique cation-anion connectivity, e.g., AB₄X₈ lacunar spinels (as shown in FIG. 3) and ABX3 perovskites where the l:m:n ratio in AlBmXn family defines the stoichiometry. Chemical composition refers to atomic identities, i.e., elements from the periodic table. Different compounds exhibit distinct structural distortions (e.g., Jahn-Teller type or Peirels-like) that lead to MITs behavior. The high sensitivity to small design changes and different microscopic mechanisms pose the hurdle in decoding a structural genome that governs the transitions. With existing technology it takes years to discover a new material since the focus is on a single material at a time where the composition is perturbed to assess if it leads to MITs.

Further, advanced density functional theory (DFT) to study the electronic structure of materials is a computational bottleneck in discovery. Such simulation takes hours to days each but are far more accurate than standard high-throughput methods which often (>90%) give incorrect predictions. Existing ML technology surrogating DFT requires many (>10⁴) simulations to achieve high accuracy, partly because ML and optimization are treated as two separate processes. Also, these methods do not guarantee synthesis feasibility; many targeted materials are likely metastable phases requiring kinetic-controlled synthesis that result in complex phase diagrams. For MITs materials, literature-reported syntheses are limited.

Materials design is a mixed-variable design problem with predominantly qualitative variables (y) such as compound family, stoichiometry, composition, and synthesis type and procedure, in addition to quantitative variables (x) such as operating pressure, stress, and carrier density. The mixed-variable nature renders surrogating and the search for optimal designs challenging. Limited ML techniques can achieve sufficient accuracy for mixed-variable input spaces where design objective f(x, y) and constraints g(x, y) are functions related to performance, cost, or synthesizability. While some f and g are computed from high-fidelity DFT simulations (e.g., reaction energy, band gap changes), others are exogenous responses from less complex calculations (cost), domain models (semi-classical transport), or ML models from literature data (resistivity), posing the need for a framework that can accommodate multi-source and multi-fidelity evaluations.

Although software packages are being developed specifically for materials design by companies such as Citrine Informatics, Lumiant Corporation, Exabyte, and Ansatz Al, they are mostly focused on specific applications or fields (e.g., specific compounds), employ off-the-shelf ML models separate from optimization, ignore systematic uncertainty quantification, mostly handle quantitative inputs, and not meant for supporting conceptual design.

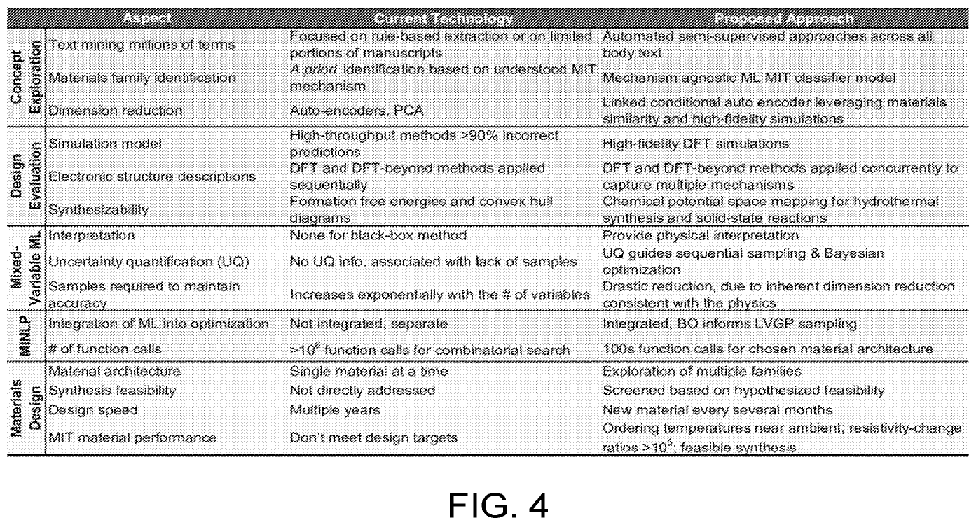

The approach utilized in certain embodiments of the present invention has the following five major innovative aspects with target performance, summarized in a table as shown in FIG. 4, in comparison to the existing technologies.
NLP and ML Assisted Concept Exploration (CE): Different from existing efforts restricted to one material family without considering synthesis feasibility, the combined use of VS and ML-assisted CE will cast a broad net over the published literature to identify synthesizable candidate material families by leveraging CVAEs and other ML techniques to generate both synthesis and property predictions. The conditioning parameters in CVAE will be modified in an AD engine (see FIG. 1C) to incorporate physical knowledge.
Multi-fidelity Design Evaluation: Through AD, the work will achieve a balance between high-fidelity simulations and inexpensive ML models for design evaluations. For MITs, both high-level DFT and “beyond-DFT” methods will be used to determine if a compound is MITs active, the quality of the transition (approximate ordering temperature and resistivity ratio), and its synthesizability. Given the range of candidate families obtained from CE, multiple microscopic MITs mechanisms will be assessed concurrently on equal footing. This multi-fidelity evaluation is rarely pursued in the materials physics domain.
Mixed-Variable Machine Learning: While most Gaussian Process (GP) models are designed for only quantitative inputs x, the novel LVGP approach being utilized can model both qualitative and quantitative inputs by exploiting the universal fact that in any physics-based simulation the categorical inputs' effects on a response must always be due to some underlying quantitative variables that are generally unknown or too high-dimensional to model directly. LVGP automatically discovers the qualitative-to-quantitative LV mapping; providing a physics-based dimension reduction, drastically reducing the number of simulations required for high accuracy.
Bayesian Statistical Representation enabling Bayesian Reinforcement Learning: The LVGP approach provides a principled Bayesian statistical representation of design responses (f and g) and uncertainty, which is a critical component in the sequential sampling of Bayesian Reinforcement Learning and Exploration. With LVGP-based BO, the approach will reduce the evaluation of millions of designs to only hundreds for a chosen family.
Expedited Materials Innovation: the inventors expect to reduce the multi-year design cycle time to six months or less. To ensure efficient functional MITs materials discovery and realization, the project will (1) Enable MITs temperatures near room temperature; (2) Realize materials with significant resistivity-change ratios across the MITs (nominally >105); (3) Develop predictive power for the MITs threshold in parameter space for new switching (transistor) paradigms; and (4) Determine the synthesis conditions for specific MITs materials to enable platform realization. The software modules of the system will be highly integrable to materials design software and suitable for rapid technology deployment and broader applications.

The potential impact of the proposed approach is significant. Electronic materials and devices have transformed technology, science, and the way human beings live. Much of this revolution was enabled by six decades of improvements in the performance of silicon field effect transistors. The approach is disruptive in that it could revolutionize microelectronics beyond today's conventional roadmaps where scaling is approaching its physical and economic limits, while the growth of data-centric computing and sensor networks redefining computing workloads is exploding. The MITs materials testbed will directly impact DOE interests in future low-power microelectronic systems, a national priority as they can deliver beyond binary switching; redefining computing and reimagining information flow as recently cited in the DOE “Basic Research Needs for Microelectronics Brochure”. Next-generation electronic materials will secure the future U.S. economy and enhance its (civilian and military) energy security and efficiency. Worldwide semiconductor revenue totaled $476.7 billion in 2018, a 13.4 percent increase from 2017, with memory technologies such as DRAM accounting for 34.8 percent of total semiconductor revenue. Samsung Electronics continues as the leading DRAM vendor and specific memory technologies that will be impacted by MITs include the superior resistive random access memory (RRAM), for which Samsung Electronics already leverages sub-optimal MITs phase change systems, and the selector materials in Intel's commercial Optane Technology™, which exploits resistive changes within the non-volatile 3D XPoint Memory Media stack.

From ML methodological standpoint, the approach is disruptive in its ability to hand combinatorial design alternatives under various conceptual configurations. Besides the novel mixed-variable ML approach via LVGP, the effort also represents one of the rare attempts in integrating text-based virtual screening with DFT based composition optimization to consider many possibilities while ensuring synthesis feasibility in design of inorganic materials. The approach utilized in certain embodiments of the present invention also applies to other energy materials design and engineering problems where mixed variables co-exist and/or co-design of processing and structure is critical.

In certain embodiments, the approach has been demonstrated and validated using the testbed of functional materials exhibiting MITs. Specifically, the approach is structured around developing the five ML-based computational modules as shown in FIGS. 1A and 1B and the Adaptive Discovery strategy linking the different modules.

### Natural Language Processing (NLP) Based Virtual Screening (VS)

To develop the knowledge base for this work and collect the initial data needed to screen for promising MITs compounds, the inventors will leverage NLP approaches to extract information from the unstructured, free flowing language of relevant scientific text. These approaches have proven valuable for some time in the biomedical discipline to obtain information about diseases for diagnosis and drug development, with more recent application to the chemical discipline. Use of NLP in materials science is a more recent endeavor and PI Olivetti at MIT has modified these previous approaches to extract information focused on material synthesis for inorganic materials. These data have been extracted with accuracies that are competitive with current state-of-the-art but on more complex classification problems (e.g., classifying across 20 different types of tokens used in scientific language at the same accuracy as binary classification models). Moreover, there are only preliminary efforts at extracting property information within materials-science texts.

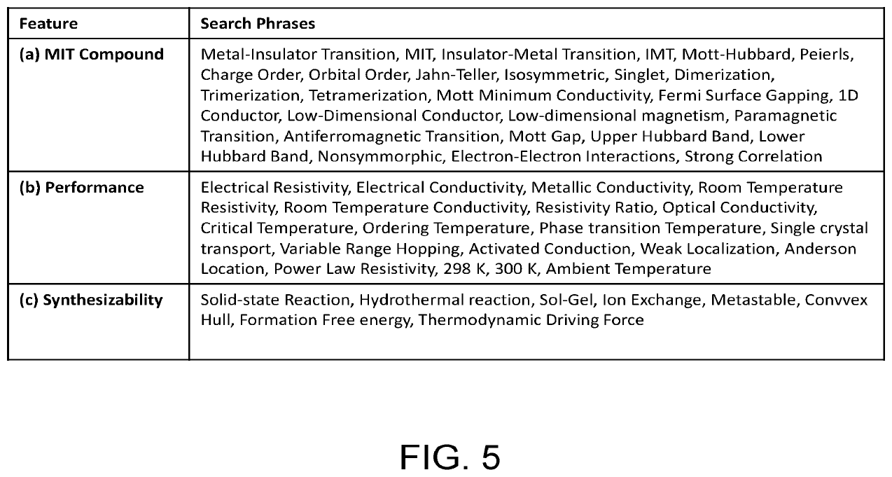

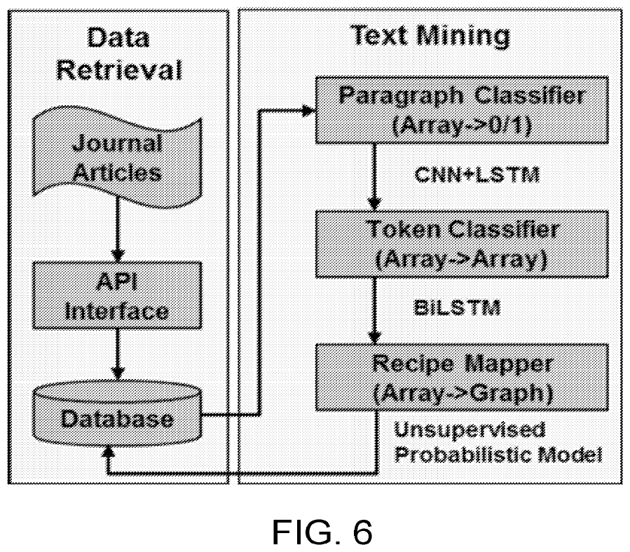

The planned implementation for MITs materials leverages the previously developed broad search capabilities across a few million manuscripts from twelve publishers to find journal articles which focus on (i) known MITs compounds, (ii) unidentified potential MITs compounds with shared (chemical, compositional, etc.) similarities, and (iii) non-MITs materials (determined exclusively to be metals or insulators), or some combination thereof. Each type of article has information that must be extracted to identify key descriptors and latent variables for subsequent exploration, evaluation, and optimization including: (a) definitive assignment of the material as MITs compounds; (b) its performance (i.e., on/off ratio and transition temperature); and (c) synthesizability. To that end, NLP will digest the literature using the preliminary list of words and phrases in the table as shown in FIG. 5. FIG. 6 schematically shows text extraction workflow of the VS module according to certain embodiments of the present invention. Specifically, the overall information extraction workflow of the VS module as shown in FIG. 6 is based on the inventors' past success with NLP applied to inorganic materials. The inventors acquire content from relevant journal articles through the CrossRef API and journal-based article downloading. Further, the inventors emphasize retrieval of HTML or XML documents and have publisher-specific parsers to guarantee that the downstream text is clean and section as well as subsection headers can be retained for later use, which helps with activities such as para graph classification.

The inventors identify and classify desired text portions. For example, using an unsupervised topic modeling algorithm such as latent Dirichlet allocation, the inventors can cluster keywords over thousands of papers and group relevant synonyms, such as electrical resistivity, phase transition temperature or resistivity ratio as relevant terms for MITs. Once the paragraphs of interest have been identified for either MITs compounds, performance, or synthesis, the words within the sentences are tokenized and then these tokens are labeled along the words of interest for identifying MITs materials (and nonMITs materials) (Subtask 1.3). Both the paragraph and token classification steps leverage word embeddings. FastText and ELMo are word embedding models that project words into m dimensional vectors and both can represent previously unseen words. To this end, FastText leverages substring-based representations (e.g., “GaV₄S₈” contains substrings “Ga” and “V₄S₈”) while ELMo is entirely character-based. The embeddings add unsupervised information to the classification model and, in turn, less manually labeled data is needed.

### Machine Learning (ML) Assisted Concept Exploration (CE)

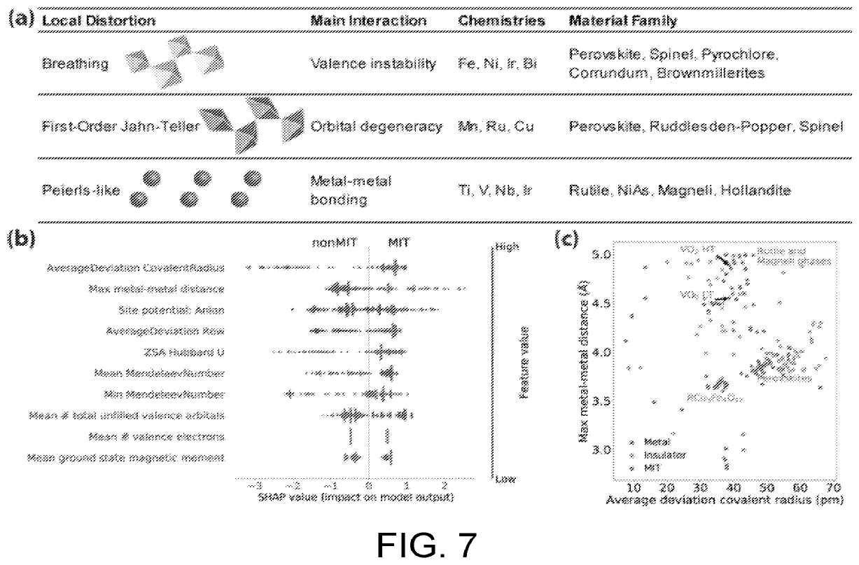

Autonomous methods to accelerate identification and optimization processes are unavailable for inorganic MITs materials. MITs arise from multiple (possibly competing) mechanisms (e.g., a purely electronic Mott transition from correlated metal to Mott-Hubbard insulator) or are accompanied by crystal-symmetry breaking (e.g. breathing, Jahn-Teller, or Peierls-like distortions), as shown in FIG. 5(a). Thus, MITs are typically found in select material families. Although there are large databases of synthesized and predicted inorganic materials (e.g. the ICSD, Materials Project, OQMD, and AFLOW) that could be theoretically screened for potential thermal driven MITs candidates, no tool exists to perform CE, the process defined as using the relevant chemistry, materials science, and physics corpus of knowledge to learn what defines MITs materials family with functionality f and constraints g for exploitation in an Adaptive Discovery cycle. To that end, CE will be pursued using ML assisted strategies, which involve DoE, CVAE, active learning, and the construction of classification and regression models of g. Outcomes of the VS task using NLP of the available corpus will feed material descriptors, keywords, and material families (including composition and structure) from which the ML models will learn what defines a MITs material from a non-MITs material, leveraging available and newly identified descriptors from CVAE deep learning feature extraction. Since not all materials will be of a known type, both supervised and unsupervised learning will be utilized to ensure the outcomes of CE task reliable, including compound identification and synthesis models. Models for MITs Material Identification. Initial DoE and CVAE Deep Learning will focus on MITs feature extraction (Subtask 2.1), which will be used in the construction of classification and regression models (Subtask 2.2) for filtering of candidate MITs materials text-mined. The proof-of-concept MITs Classification Model as shown in FIG. 7(b) was constructed using a small materials dataset informed by domain science knowledge (roughly 200 compounds) and a few additional domain-science features with gradient boosted decision trees. Importantly, previous efforts have successfully built ML classifiers for separating compounds that are exclusively metals or insulators, whereas the inventors constructed the first ever MITs classification model. Compared with the aforementioned published classifiers, the model's metrics are comparable (median receiver operating characteristic ROC area under curve=0.91/0.96 with interquartile ranges 0.12/0.09 for the metal and insulator classifiers, respectively) and leverage the newly built features (as shown in FIGS. 7(b), (c)). Although the model comparison is not exact, it serves as a crucial demonstration of the inventors' capability to differentiate MITs and non-MITs materials with ML.

### Active Learning of MITs Performance.

The predictive models are incorporated for exogenous constraint functions g (e.g., room-temperature resistivity prediction and transition temperature estimation) for design optimization of MITs performance parameters (Subtask 2.3), which are not direct outputs of the DFT simulations: Both models will be constructed using active learning of the available experimental literature and NLP methods to quickly and robustly build suitable models. In active learning, a model selects which instances for which to label a g value to maximize the accuracy of the fitted model for a fixed sample size. Criteria for selecting the next sample to label falls into two categories: informativeness (how uncertain the classifier candidates are on the selected training instance or how much the candidates disagree with each other) and representativeness (how diverse the chosen samples are or whether the distribution of selected samples is like the testing data distribution) expected error reduction. Because some of the types of information needed for the downstream activities are not only within the body text, but also within figures (i.e., those in the item (b) of the table as shown in FIG. 5 for existing MITs materials), this active learning approach focuses effort for annotation on the richest set of figure-based information.

### Deep Learning Synthesizability.

Starting from the natural language text extracted by the VS module, the next step is to apply word embeddings from language models and feed them into a named entity recognition model, upon which a CVAE will be trained to generate syntheses procedures for any MITs compound independent of materials family. The inventors will predict precursors, reaction types, and synthesis actions (time, temperature, pH, etc.) for existing materials leveraging the recent demonstration of the capability applied to complex perovskite oxides. The model will learn representations of MITs materials corresponding to synthesis-related properties, and the underlying phase transition mechanism will be understood using existing and thermodynamic knowledge aided with first-principles free energy calculations. The generative aspect of the CVAE model will be further elaborated on Adaptive Discovery.

### Incorporation of Exogenous Regression Models.

Proposed MITs materials identified in this project have the potential for high-volume use by the microelectronics and semiconductors if they meet the performance demands. For adoption of new materials platforms, the compounds should be composed of elements that are accessible and not in danger of a supply risk. To that end, the Mixed-Integer Optimization task will be fed exogenous performance and cost-based f and g models (Subtask 2.5), e.g., market concentration based on the Herfindahl-Hirschman indices (HHI), elemental scarcity, and embodied energy, which are practical materials-platform issues that influence widespread technology adoption of any optimized MITs material discovered in this project.

### Latent Variable Gaussian Process (LVGP) Modeling for Mixed-Variable ML

Once the potential MITs families are generated from CE, a novel LVGP approach is proposed in this work to create ML models for physics-based high-fidelity simulations with mixed-variable inputs. There are a few existing ML models that can handle mixed variables as inputs such as neural networks or random forests and boosted trees. However, GPs are ideally suited to the problem for a number of reasons that also underlie their popularity for surrogating physics-based simulations. First, contrary to most other ML models, GPs interpolate the data and hence are ideal for surrogating deterministic responses like DFTs. They can also conveniently handle simulations with stochastic outputs via the nugget parameter. Second, they provide a principled Bayesian statistical representation of (f|x, y) (i.e., both f and its uncertainty distribution) which enables data fusion once integrated with the CVAE modeling in Section 2.1.1. Lastly, GPs provide closed form conditional distributions which are essential for the reinforcement learning parts of the BO in Section 2.1.4. However, most GPs are designed for only numerical inputs, x, and the associated correlation functions cannot handle qualitative factors, y. A few covariance structures have been proposed for mixed-variable GP but they are generally ineffective given the use of simplified parameterizations that are unable to accurately represent the underlying physics.

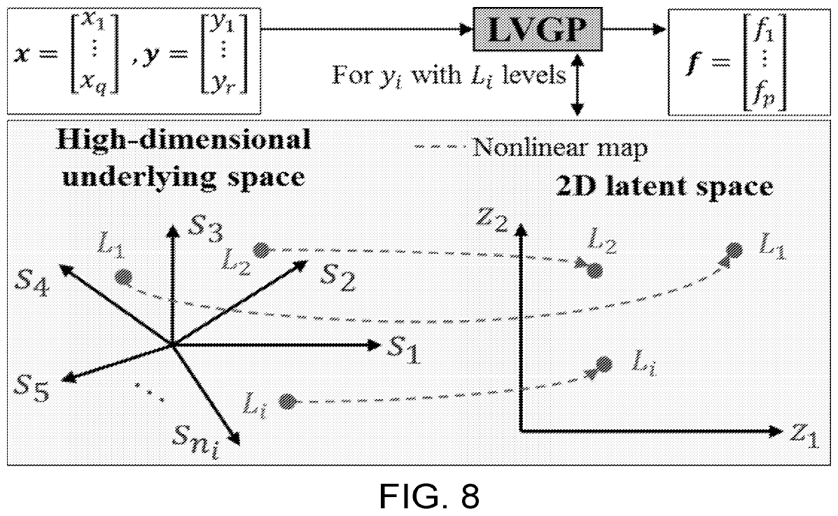

The inventors have recently proposed a fundamentally different method that involves a latent variable (LV) representation of qualitative inputs. The main idea is to map the levels of each yi to a set of numerical values for some underlying latent unobservable quantitative variable(s) z, as shown in FIG. 8. Since underlying numerical variables are generally unknown or too high-dimensional, the LVGP approach automatically discovers a categorical-to-numerical nonlinear map that transforms the underlying high dimensional physical attributes of each yi into the LV space. This mapping has strong physical justification as the effects of categorical inputs in any physics-based simulation model must always be due to some underlying numerical variables. As opposed to other ML methods (linear like PCA or nonlinear such as manifold learning, autoencoders, and restricted Boltzmann machines), the nonlinear mapping also provides an inherent ordering and structure for the levels of the factor(s), which leads to substantial insight into the effects of qualitative factors. In essence, the LVGP modeling is a form of dimension reduction that incorporates the crucial information from the physics-based simulation about the effect of the variables on the response which is in contrast to unsupervised methods that use multi-response covariances.

With the mapping, the covariance model over (x, y) can be any standard GP covariance model for quantitative variables over (x, (y)), e.g., the Gaussian correlation function:

$$( f ( x , y ) , f ( {x}^{′} , {y}^{′} ) ) = exp \{ - {\sum }^{q} {ϕ {( x - {x}^{′} )}^{2}}_{i = 1 i i i} - {{‖ z ( y ) - z ( {y}^{′} ) ‖}^{2}}_{2} \} , \qquad ( 1 )$$

where z(y) is the numerical vector of mapped LVs and |⋅|₂ denotes the Euclidean 2-norm. The mapped values {z(y)} and all hyperparameters can be obtained in a straightforward and computationally stable manner via maximum likelihood estimation (MLE) where the total number of estimates,

$$q + 2 ( {\sum }_{i = 1}^{r} {m}_{i} - 1 ) ,$$

is linearly proportional to the dimension of x and the number of levels (mᵢ) of each yi, significantly reducing the samples needed compared to exiting methods.

As a proof-of concept, the inventors have shown the superior performance of LVGP over existing approaches for problems with both large or small number of qualitative variables. The method will be further enhanced under Task 4 (Subtasks 4.1-4.4). The approach will also be integrated with BO for material design, introduced next.

Mixed-Variable LVGP based Bayesian Optimization (BO) for MINLP While most existing mixed-integer nonlinear programming (MINLP) algorithms are effective for inexpensive (x, y), they are not suitable for the problem where each high fidelity physics-based DFT simulation costs hours to days. Recently, there is a growing recognition that BO provides an effective materials design optimization framework; it offers an adaptive paradigm to efficiently sample the design space and identify the global optimum while utilizing data from various database and treating existing knowledge as “starting point”. Nevertheless, existing materials design applications of BO are all limited to continuous design variables due to the lack of Bayesian inference model for qualitative variables or mixed-variables.

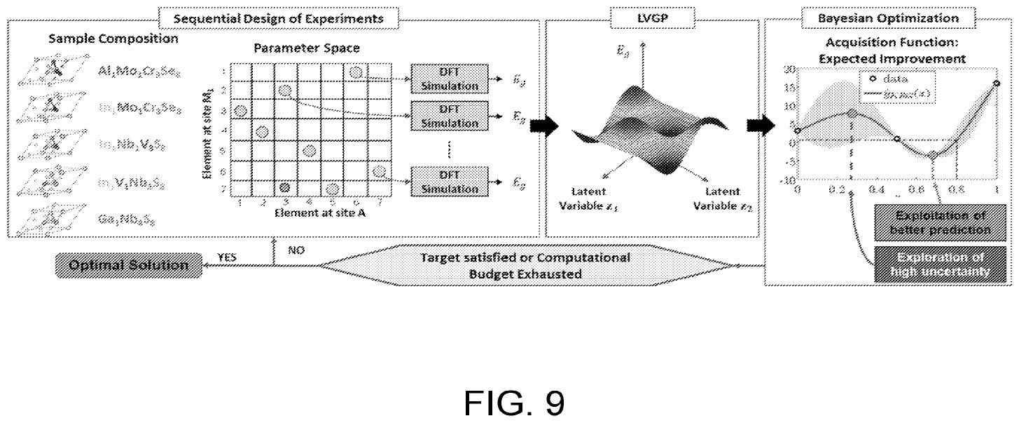

These limitations can be addressed by integrating the novel LVGP model into the BO framework. As described, LVGP provides a probabilistic representation of (x, y) which is a critical component in BO and principled exploration (i.e., within a formal statistical framework, i.e., reinforcement learning). As shown in FIG. 9, using the prediction uncertainty quantified by the LVGP model built using current data, the approach follows an adaptive sampling procedure to select new (x, y) combinations to run high-fidelity simulations, where the acquisition function is used to balance between exploitation (zeroing in on the optimal design) versus exploration (sampling where uncertainty is large), to achieve global optimality at the end.

As a proof-of-concept, the inventors tested the feasibility of LVGP-based BO on designing emerging materials systems for simultaneous composition selection and microstructure optimization of a solar cell, design of optimal hybrid organic-inorganic perovskites, and concurrent material constitute, microstructure, and surface treatment design of nanodielectric polymers.

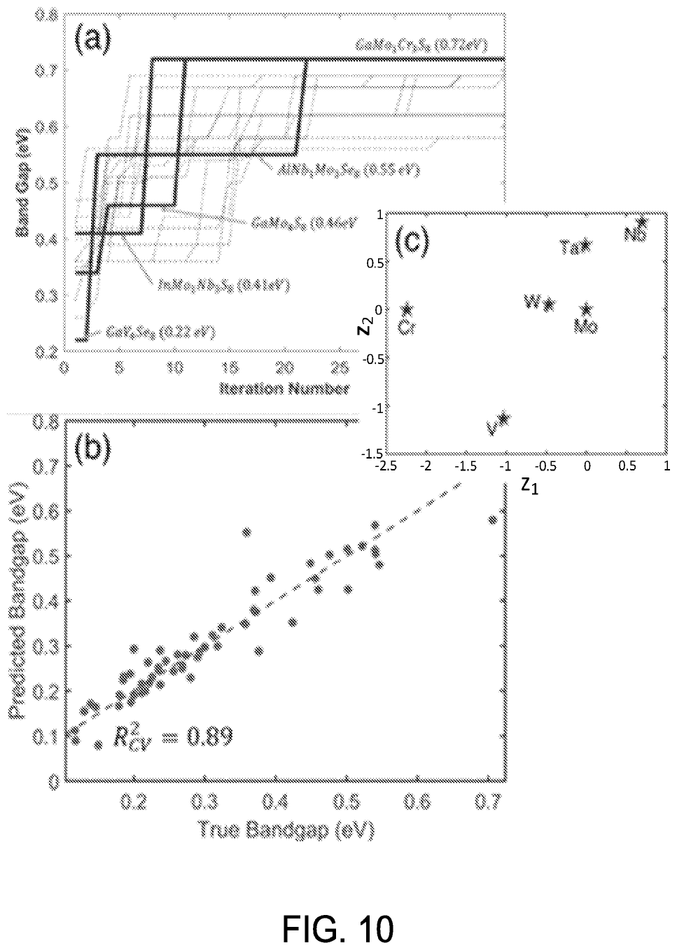

The inventors have also recently applied a single-objective LVGP based BO process to a successful MITs materials discovery: A new lacunar spinel, GaMoCr₃S₈, with a maximized band gap, as shown in FIG. 10(a), was achieved by DFT evaluations of less than 7% of the total search space of 324 candidate materials within the chalcogenide lacunar spinel architecture with composition GaMo₄S₈. The predicted band gap compared with their true values from DFT demonstrates the high accuracy of the LVGP model in learning composition property relationships for this MITs architecture as shown in FIG. 10(b). The latent space representation reveals clustering of Mo, Nb, and Ta, as shown in FIG. 10(c), indicating these cations play a similar role in the MITs, bringing physical understanding.

The approach will be generalized to multiple, possibly functional responses associated with MITs design objectives f and constraints g. The inventors will extend the method to include multiobjective BO and synthesizability in constraints (Subtask 5.1), some simulated from DFT and some assessed using the ML models from CE or exogenous analysis. The inventors will also generalize the LVGP-based BO to reinforcement learning (Subtask 5.2) where, instead of sampling one point at a time in the traditional BO, a sequence of samples (called policy) will be determined to maximize the reward function. Finally, the results from LVGP and BO will assist Adaptive Discovery.

### Adaptive Discovery: Iterative Simulation, Exploration, and MV Optimization.

The AD loop encompasses three blocks (DFT simulation, LVGP modeling, and BO) that will be iteratively and sequentially executed, with the purpose of adaptively exploring the input space to optimize the design and discover new materials families (architectures) that possess the desired properties. The CVAE in CE provides generative distributions like (x, y)=∫(x, y|v)(v)dv, which characterizes the distribution of (x, y) from the existing literature. In contrast, the LVGP modeling in the AD loop provides conditional distributions like (f|x, y), which represents the state of knowledge from the DFT and other sources on the functional dependence of f on (x, y). To characterize and explore the promising input (x, y) regions and inform the BO, the inventors will integrate these two types of information via a Bayesian framework and computations to produce generative conditional distributions like p(x, y|f=f*)=p(f=f*|x, y)p(x, y)/p(f=f*), which is interpreted as a generative distribution for producing candidate (x, y) that are likely to result in some desired value f* for the response f. To simplify notation here, the inventors focus on a single f, but the methods will generalize to computing quantities like (x, y|a≤f≤b) (to target f over a range of values), (x, y|f=f*, a≤g≤b) (to target a specific value for f while simultaneously satisfying inequality constraints on g), etc. The knowledge of (f|x, y) typically be sparse and incomplete at the end of VS and CE stages, but will be continuously refined during AD via the sequential DFT simulation and mathematical modeling.

The AD begins by simulating the property fᵢ for each (xᵢ, yᵢ) from the initial DoE. It then adaptively explores new designs by iteratively generating new (xᵢ, yᵢ), simulating their properties via DFT, using LVGP to update (f|x, y), which provides a predictive model for f and its uncertainty (see Task 4), and then BO to guide and optimize the adaptive selection of next inputs (xᵢ, yᵢ)(see Tasks 5 and 6). As described under Task 6, the inventors will use Bayesian modeling and computational methods to incorporate other information from the VS CVAE and CE active learning. While the AD loop begins by exploring the (x, y) space spanned by the existing designs that have been considered in the prior literature, it will eventually move to exploring the broader (x, y) space, encompassing substantially different families/architectures to be discovered.

There are no major risks expected on the technical side due to the completed proof-of-concept of key technical components of this proposal, the initial success of using the approach for designing novel MITs materials, and the established records of key personnel on this team. The inventors have identified a few potential risks and suitable responses. For example, to address the risk of insufficient access to relevant journals, the inventors will leverage the existing database and add more publishers if necessary. A potential risk of using the LVGP is the accuracy in fitting hyperparameters for large dimensionality (many categorical variables with numerous levels). The inventors will mitigate the risk by extending the conventional GP models' robust fitting approach.

Although first-principles quantum mechanical calculations have become a vital tool for the discovery, design, and development of new functional materials, one challenge in the DFT HFE is due to limitations of standard exchange-correlation potentials (Vxc) to produce reasonable band gaps upon the MITs. To mitigate this risk, “beyond-LDA” techniques, starting with Hubbard U approach, kinetic-energy density functionals, and hybrid-Vxc will be applied. The plus-U method is the most computationally feasible means to reproduce the correct ground states in Mott insulators. In addition, SCAN has recently been shown to provide complete descriptions of MITs without requiring dynamical mean field theory methods. The inventors have extensive experience with these functionals and approaches. Another tactical computational risk involves availability of compute-cycles for the DFT evaluations in the project. The inventors will leverage experience in accessing DOE high-performance-compute (HPC) resources, e.g., NERSC with multi-million cycles annually, to ensure sufficient computational time is available for high-fidelity simulations. The inventors will also expand the HPC capacity at Northwestern by purchasing nodes for exclusive use on this project.

### Example 1

In the following example, the five ML-based computational software modules of the system as shown in FIGS. 1A and 1B for the approach has been demonstrated and validated in different tasks using the testbed of functional materials exhibiting MITs.

### Task 1: Virtual Screening

This task takes the inputs from published journal papers and patents as well as search criteria for MITs compounds, and then generates outcomes including (1) a set of candidate MITs materials and their synthesis parameters, (2) key words, frequently used words in existing papers on MITs materials, and (3) all existing MITs materials along with their relevant properties.

### Subtask 1.1: Problem Definition.

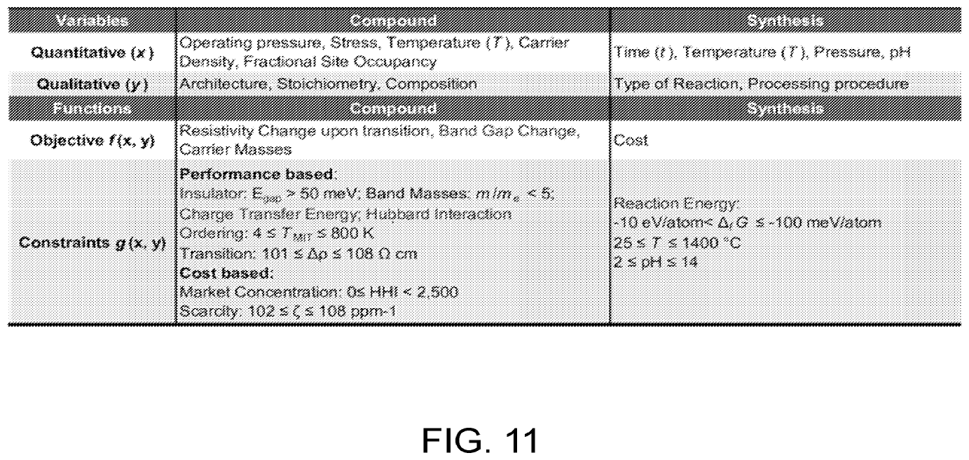

Task outcomes include desired materials functions and properties, physical mechanisms, and optimization formulation-aspects critical for implementing the proposed framework. FIG. 11 shows a table of examples of (x,y) variables associated with MITs compounds and synthesis processes, respectively, and initial design objective (x, y) and constraint g(x,y) functions. While some f and g are computed from high-fidelity DFT simulations (indicated in blue), some others are exogenous responses from simple calculations (cost), domain science (transport) models, or ML models created with literature data (e.g., finite temperature resistivity) under Task 2.

### Subtask 1.2: Collection of Relevant Texts.

This task will populate a database with relevant articles from a broad range of publishers. The current publishers being used include RSC, Elsevier, IEEE, Springer, Wiley, ECS, Taylor and Francis, APS, IOP, and ACS and the inventors have individual publisher agreements with each of these publishers that enable us to perform Text & Data mining activities. The inventors focus on HTML or XML documents but can also intake PDFs as needed.

Subtask 1.3: Information Extraction from Texts.

To obtain the relevant information from the text this task requires 1) entity recognition through tokenization of relevant text which can be processed both individually or as a sequence, normalization to lemmatize a term, part-of-speech tagging using dependency parsers (which will be developed specific to the MITs domain as needed), and token classification; 2) entity linking, which includes mapping recognized entities to relevant information within the developed knowledge base (Table 2); and 3) relation extraction, which includes the relevant connections between entities, e.g., between an MIT compound and its resistivity.

### Task 2: ML Assisted Concept Exploration (CE)

Unsupervised learning methods will be used to link scientific literature to MITs material insights by extracting features. Supervised and adaptive learning schemes will be used to build models in Subtasks 2.2-2.4, enabling learning the x and y that define MITs material representation and performance. Exogenous regression models will also be formulated to ascertain materials costs and assess likelihood of adoption of the material by industry. The main outcomes include candidate materials families, synthesis recipes of candidate materials, learnable constraint functions (of x and y) describing MITs materials.

### Subtask 2.1 Initial DoE & CVAE Deep Learning Feature Extraction.

This task will acquire important data from natural text as input for Task 2 models. Following data discovery and collection, the inventors will structure, clean, merge, and augment the data. The inventors will also map the entities and their relations in a VAE architecture to identify the relevant generative latent space to identify potential candidate families. Visualization of the corpus will enable identification of the key tokens for MITs as well as decoding the late space. Outcomes include capturing all known MITs and non-MITs compounds (including unidentified ones) to be used for subsequent model training and validation. This task will feed into classification models through redefine/redesign of search criteria and updated features since CVAE provides a vector of popular elements (e.g., early transition metals), frequently appearing material families (e.g., spinels), and conditions of transition (e.g., stress).

### Subtask 2.2 Classification Models for Candidate Material Families.

Key tasks to construct an improved MITs Classifier require augmenting the existing dataset of compositions and structures with MITs and relevant non-MITs compounds extracted from the natural language text. Additional inputs include (1) a set of raw candidate MITs materials and possible synthesis parameters (from Subtask 2.1), (2) latent space representation of existing MITs materials, (3) frequently used keywords in existing papers on MITs materials, (4) all existing MITs materials, and (5) relevant non-MITs materials from which the model can learn. Materials with known thermal MITs will be labeled and featurized with new domain-science features and embeddings, from Subtask 2.1. Metals and insulators will be then defined using room temperature resistivity of 1 Ωcm as the cutoff. Supervising learning schemes will be used to build the classifier using various cross-validation and first-principles calculations for performance assessment. Task outcomes include refined candidate material families for DFT which will be fed into the BO machine (with LVs from CVAE) to build a better generative model.

Subtask 2.3 Active Learning of MITs Responses with Regression Models.

Active learning approaches will be used to extract physical data from the literature, temperature-dependent resistivity data that is usually reported in graphical format, to construct a regression model that predicts the resistivity of a material at a specified temperature (e.g., room temperature). This approach is relevant for the data that prove challenging to extract from the literature. Here the inventors provide manual annotation information to an optimization model that has access to the texts the inventors are extracting from in order to identify potentially rich areas for further extraction. This constraint model will use as inputs material family, stoichiometry, and composition, and use features from Subtask 2.1 and 2.2 to make predictions. The model will be validated using experimental data heldout of the training set and first-principles based f functions (see the table in FIG. 11) coupled with semi-classical transport theory. A similar scheme will be used to build an ordering temperature TMIT model.

### Subtask 2.4 CVAE Deep Learning for Generating Synthesis Recipes.

Synthesis recipes will be generated for MITs materials using a CVAE method, whereby reagents, reaction type, and synthesis actions are specified. The model will be validated using training data published over a decade prior to their first reported syntheses of a test material. The model will be used to perform synthesizability screening for proposed MITs materials and requires an MITs-tuned CVAE architecture to include the compounds from the literature and the vector associated with their synthesis procedure, which includes precursors, operations and conditions. Aspects of the synthesis procedure related to formation of the targeted product will be assessed using DFT calculations with first principles based thermodynamic simulations. Embodied energy costs due to the processing procedure will also be computed to assess cost and technology feasibility of the MITs material.

### Subtask 2.5 Exogenous Regression Models for MITs Cost and Performance.

The Herfindahl-Hirschman index (HHI) measures market concentration using geological data (elemental reserves) and geopolitical data (elemental production) for the periodic table. HHI will be calculated for elemental production (HHIP) and for elemental reserves (HHIR), using known deposits using the USGS commodity statistics. HHI for the materials will be based on weight fraction of elements in the chemical formula. A similar model will be created to describe material scarcity,(, beginning with crustal abundance of the elements and processing costs, including embodied energy evaluations, which is the sum of all the energy required to produce material. The inventors will then construct exogenous regression models to learn the x and y representation of MITs compounds.

### Task 3: DFT Based High-Fidelity MITs Evaluations

This task will employ plane-wave DFT as implemented into the Vienna Abinitio Software Package (VASP) to perform the computational assessments involved with MITs compound evaluation, which are formulated as three subtasks. Outcomes of these evaluations will feed further optimizations as well as deliver a database of materials of use for catalysis and energy generation, storage, and conversion technologies as MITs compounds exhibit chemistry compatible with multiple oxidations as in redox processing active in the aforementioned technologies.
Subtask 3.1 Computational MITs compound identification requires the material exhibits (at least) two phases of the material (polymorphs)—the metallic and insulating states. Based on the function optimization, the insulating state will be identified by the LVGP+BO task; therefore, DFT calculations will be performed to ascertain the origin of the electronic band gap and determine what role atomic structure plays in producing the gap. This understanding will be quantified using group theoretical methods and enable identification of the corresponding metallic state upon which additional DFT calculations will be performed. The inventors will compute the electronic band dispersions, macroscopic dielectric properties and electric/magnetic polarization of each phase.
Subtask 3.2 Assessing the MITs mechanism and responses requires that the primary and secondary structural mechanism contributing to the stabilization of the ground state are extracted. To that end, the inventors will leverage mode-crystallography of the distorted structures. The different roles played by these modes will be isolated by means of DFT calculations of the energy landscape. This analysis will enable identification of the important electron-lattice interactions by evaluating the shifts (and changes in hybridization) in the electronic orbital levels with respect to the symmetry modes and discern the MITs mechanism. These energy scales and interactions will be used to approximate MITs transition temperatures and evaluate approximate resistivity values at the level of semi-classical transport theory. The latter will be compared to ML-models of resistivity. This approach was successfully implemented to drive MITs in the past.
Subtask 3.3 Synthesizability metrics will also be DFT calculated total and formation free energies using convex hull approaches and chemical potential maps to guide hydrothermal synthesis and solid-state reactions. Energetics from the DFT simulations will also integrated with the newly implemented NLP scheme to construct solid-state synthesis and processing maps to facilitate the ultimate realization of the predicted materials.

### Task 4: LVGP for Mixed-Variable Machine Learning

The goal of this task is to further validate and enhance the novel LVGP approach for high-dimensional responses, high-dimensional inputs and data sets, active learning of exogenous responses from Concept Exploration, and to implement the method for the MITs testbed.

### Subtask 4.1 Multi-and-Functional Response LVGP.

Complex computer simulations generally provide high-dimensional responses, i.e., multiple scalar outputs and/or (multiple) functionals, e.g., band gap, number of states at the Fermi level, electronic band widths, or crystal structure information with local structural parameters correlated with resistivity and the MITs transition temperature. The inventors will employ all DFT responses in LVGP modeling to leverage the crossresponse correlations and, in turn, enhance LVGP's predictive power. The inventors will first use nonlinear manifold learning to characterize functional responses such as resistivity with a few physically meaningful scalars. Then, the inventors will re-formulate LVGP to have a separable covariance function to apply it to the collection of the original scalar responses and the scalar responses from manifold learning.

### Subtask 4.2 Collection of DFT Training and Validation Data for LVGP Modeling.

Training data (˜10² for each family) for MITs descriptors and materials stability will be generated from high-fidelity DFT (Task 3) for both property predictions and the calculation of relevant energies for assessing phase (meta)stability. The latter will be used in the CVAE (Task 2) to ensure that material predictions are made on viable synthetic candidates simultaneous with synthetic processing conditions. Cross validation will be used to split the training and validation data sets. Since the physics governing the MITs in different candidate families is rather different, the LVGP models will be constructed from calculated DFT data within multiple material architectures, e.g., lacunar spinels, pyrochlores, etc., and other additional classes identified using the NLP schemes (Task 1). Models will then be used for LVGP-BO (Task 5).

### Subtask 4.3 LVGP for Large Dimensions and Datasets (Apley, Chen).

GPs are normally not suited for large data (>10⁴ samples) since building, storing, and inverting the covariance matrix becomes prohibitive. Existing methods do not employ the entire information in the data which prohibits GPs to have a similar performance as, e.g., neural networks. In a recent work, the inventors have developed a robust, intuitive, and computationally efficient method based on the convergence behavior of hyperparameter estimates that enables GP modeling for very large datasets (>10⁵ samples) which achieves the accuracy of neural networks and outperforms other GP models for large data. The inventors will extend this idea to LVGP which will involve systematic tracking of the convergence of not only the hyperparameters in the real space but also the coordinates in the latent space. The inventors will compare the approach against deep neural networks to test its predictive power.

### Task 5: Mixed-Variable LVGP Based Bayesian Optimization

Building upon the initial success of using LVGP for mixed variable BO, in this task, the inventors will further test the validity of this approach to problems with large numbers of qualitative variables and levels. The approach will be extended for multicriteria BO with constraints. In addition, the acquisition function approach will be generalized to reinforcement learning.

Subtask 5.1 Multicriteria BO with Constraints.

BO has been traditionally only applied to single objective problems without constraints. The inventors will extend the acquisition functions (AFs) for sampling in multicriteria situations. The Expected Hyper Volume Improvement (EHVI) method will be employed to combine the EHVI of the Pareto frontier and the probability for feasibility of new candidates. The inventors will often have multiple constraints (inequality and/or equality on the functions g) that must also be satisfied when optimizing f, and for this, the integrated CVAE/LVGP conditioning framework described in Task 6 will be used.

### Subtask 5.2 From AF to Generalized Reinforcement Learning (RL).

Instead of sampling one point a time using AF in the traditional BO framework, the inventors will determine a sequence of samples (called policy) to maximize the reward function. Existing BO approaches rely on short-term rewards to select the best samples while the inventors will establish a structure that particularly encourages long-term rewards. To manage the computational complexity of solving RL based BO problems, the inventors will apply latest RL algorithms such as deep Q-networks that focus on learning the value functions, and the policy-gradient-based algorithms focusing on learning the policy functions. The RL-based approaches will be tested against AF based BO methods.

### Subtask 5.3 Validation and Application to Multiple MITs Families.

In addition to testing the validity of LVGP-based BO on benchmark MINLP problems with high dimensions (>20) and many levels (>5) for each qualitative variable, the inventors will validate the approach by applying it to multiple candidate MITs families from the “Concept Exploration” module. MITs performance metrics (see the table as shown in FIG. 11) will be used to set the constraints and objectives.

### Task 6: Adaptive Discovery

The AD loop encompasses iterative DFT simulation (Task 3), LVGP modeling (Task 4), and BO (Task 5) to adaptively discover and optimize the materials' design. Each task below ensures integration in the AD loop and informed VS and CE models for adaptive updating.

### Subtask 6.1 Integration of LVGP and CVAE for Design Exploration:

The generative distributions like p(x, y|f=f*) that are computed in the AD loop will be used to directly produce promising (x, y) values to simulate and to inform the BO, the role of which is also to produce promising (x, y) values. Hence, the former will be explored as an alternative or complement to the latter. This task involves developing this framework, as well as the computational Bayesian methods to implement it, which includes variational Bayes methods common for VAEs. This task also involves developing a custom CVAE architecture and empirical model fitting that will handle conditioning on multiple f's and g's.

### Subtask 6.2 Exploration Far Beyond the Existing (x, y) Space:

The CVAE generative distribution (x, y) of Task 6.2 characterizes existing literature and allows interpolation and small extrapolation in that space. To discover radically new materials/syntheses, the inventors will extend the approach to explore outside or “orthogonal” to the existing (x, y). In its simplest form, in the expression (x, y|f=f*)=(f=f*|x, y)(x, y)/p(f=f*), the inventors take the marginalized “prior” p(x, y) to be a uniform distribution. The inventors will also investigate alternatives that place more weight (higher p(x, y)) on inputs that are more easily synthesized or, more generally, that have better properties based on exogenous f's and g's from the CE and active learning.

### Task 7: Software Development and Technology to Market (T2M)

The goal of this target is to create the data repository by leveraging the data infrastructure of CHiMaD (Center for Hierarchical Materials Design) at NU, and develop in-house software for the computational modules in the proposed framework, to be compatible with T2M.

### Subtask 7.1 In-house Implementations and Validation.

The inventors will develop in-house software packages for a. custom-architecture CVAE, b. high-fidelity DFT simulations, c. large scale and multi-response LVGP, and d. Bayesian reinforcement learning. To ensure validity, the inventors will test each package on a set of problems with a particular emphasis on MITs materials. The performance of each package will be tested against competitive approaches (if any) reported in the literature.

### Subtask 7.2 Integration and Technology to Market (T2M).

To enable fast transition from the lab to commercial deployment, the inventors will implement the methods and algorithms in open-source software packages. These packages will be extensively documented and accompanied with a visualization toolkit. The inventors will hire professional programmers to debug the codes, increase the computational speed by re-writing expensive modules in C++, seamlessly integrate the packages as shown in FIGS. 1A and 1B, and build an API to facilitate use. T2M market plan is described in Section 4.

### Example 2

In the following example, the approach has been demonstrated and validated in a 24-month project using the testbed of functional materials exhibiting MITs, with the targeted reversible resistivity changes (˜10⁵) near room temperature. At the end of the 24-month project, the inventors will deliver a series of new ML techniques using NLP, conditional variational autoencoders, active learning, latent-variable Gaussian processes, and reinforcement learning in Bayesian optimization. The project will also result in new predicted MITs compounds and improved understanding of MITs microscopic mechanisms, which in turn will revolutionize microelectronics science to provide energy-saving solutions.

### 1 Virtual Screening

The inventors successfully defined the desired materials functions, target properties, suitable material families, underlying physical mechanisms, and the mixed-integer optimization problem. The inventors also successfully used natural language processing (NLP) to curate a specialized text corpus of 70,173 papers focused on MIT materials or closely related subjects and identified key word embeddings. The inventors completed this task by further refining the search phrases to narrow down the broader paper corpus and extract relevant synthesis information for MIT materials of interest. The inventors applied the NLP pipeline to 6,000 relevant papers concerning MIT perovskite materials to extract recipe paragraphs of each paper. The inventors focused on data gathering to inform the synthesizability of MIT materials based on the synthesis information from the recipe paragraphs.

### M1.2 Relevant Texts Collected

The objective of this task was to create a highly specialized metal-insulator transition (MIT) text corpus using an initial text corpus of over 4 million scientific journal articles and papers curated by the Olivetti group. This task was initiated using a keyword list in the MIT Compound section (section (a)) of the table as shown in FIG. 5 to search through titles, abstracts and introduction paragraphs using a regular expression matching algorithm. This resulted in a specialized group of ˜70,000 papers focused on MIT materials or closely related subjects, such as materials which exhibited interesting correlated electron effects. This task was completed by further refining the search phrases to narrow down the broader paper corpus and extract relevant synthesis information for MIT materials of interest.

One promising class of materials which exhibit metal-insulator transitions is the RuddlesdenPopper (RP) perovskite family, a specific case of the general family of perovskite materials. Perovskites are compounds which are of the chemical formula ABX3 where A and B are cations and X is an anion (usually oxygen), while RP perovskites are of the chemical formula Aₙ₊₁BₙX₃ₙ₊₁ where usually A is a rare earth metal, B is a transitional metal, and X is oxygen. In order to gather additional synthesis information about perovskite materials and in particular RP perovskites, the initial text corpus of over 4 million scientific journal articles was first searched for general occurrences of perovskite materials using a list of pertinent search phrases, resulting in a group of ˜55,000 perovskite papers. This perovskite-paper corpus was then searched for specific occurrences of RP perovskites and MIT compound keywords to develop a specialized group of ˜4,000 papers concerning MIT perovskite materials. Similarly, the initial group of ˜70,000 MIT papers was searched using a keyword list relevant to perovskite materials to curate an additional specialized corpus of ˜2,000 papers relevant to MIT perovskite materials. The two specialized paper collections were then merged into a final collection of ˜6,000 relevant papers concerning MIT perovskite materials. As new literature articles continue to be published in the area of metal-insulator transition materials, the inventors will continue to update the relevant paper collections and ensure they are collected by the text extraction pipeline.

M1.3 Information Extracted from Texts

Using the specialized MIT perovskite text corpus of ˜6,000 relevant papers concerning MIT perovskite materials and the Olivetti group NLP pipeline described in M1.3, target and precursor materials were extracted from the recipe paragraphs of each paper. The Olivetti group NLP pipeline contains a machine learning (ML) augmented classifier which can automatically classify paragraphs in a scientific paper into those that most probably contain a synthesis recipe versus those that do not using a neural network and rule-based combined approach.

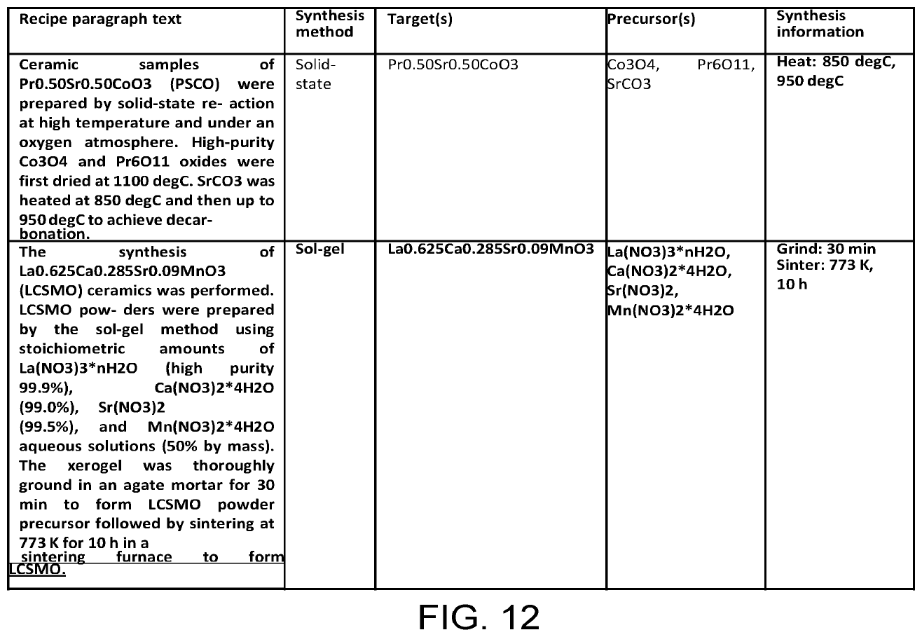

An initial effort to investigate the synthesizability of MIT materials focused on extracting relevant recipe paragraphs and synthesis information from the recipe paragraphs. Recipe paragraphs were first classified into different synthesis methods using a rule-based approach. Synthesis methods of interest included solid-state, hydrothermal, sol-gel, and ion exchange methods (as described in section (c) of the table as shown in FIG. 5) where paragraphs were searched for relevant keywords pertaining to individual synthesis methods to classify them under a synthesis method. As shown in the table of FIG. 12, the synthesis method of the recipe paragraph was determined and the relevant target(s) and precursor(s) were extracted along with relevant synthesis information. Precursors were determined using a regular expression text mining approach, where a text string containing a compound name was broken up into individual elements and a precursor was identified if the compound contained at least one element in common with the target of interest. Temperatures and times of various heating operations such as sintering, heating, calcining, etc. were the main information of focus and extracting other synthesis information will be a focus later.

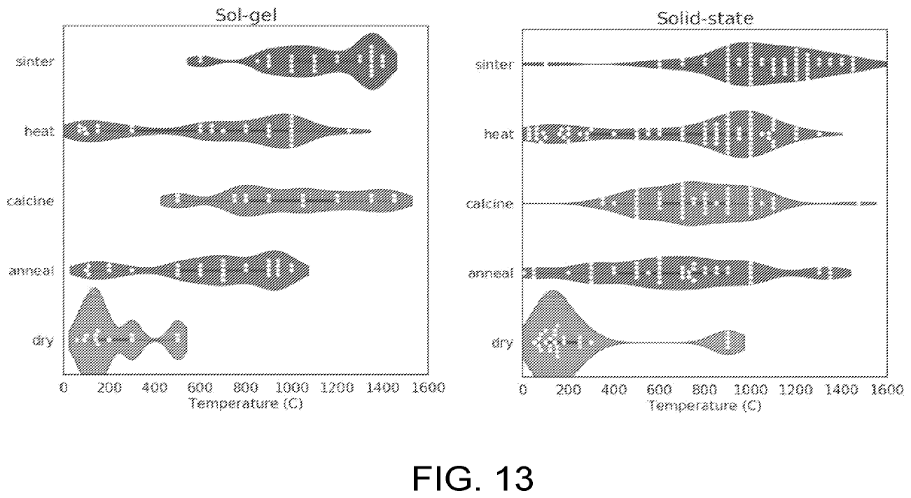

A representative plot of extracted temperatures of sol-gel and solid-state reaction synthesis methods for MIT perovskite synthesis is shown in FIG. 13 for selected heating operations. From the violin plots shown, different heat treatment methods evidently shown differences in the range of temperatures commonly employed. For instance, calcination and sintering temperatures skew towards higher temperatures while heating, drying, and annealing temperatures skews toward lower temperatures for both sol-gel and solid-state reaction synthesis methods. Moreover, calcination temperatures of extracted sol-gel synthesis tend to skew higher than of solid-state synthesis, while other operations tend to show similar distributions of temperatures between the two synthesis methods. Extracted synthesis information ultimately will be used to better understand differences in synthesis methods for metal-insulator transition materials and inform model architecture for task M2.3, either as physical inputs to the model or as guidelines for model formulation.

The future plan involves continuing to collect, refine, and clean relevant synthesis information regarding MIT materials, and in particular MIT perovskite materials. Additional data such as the identities, times, and orders of different operation steps as well as target and precursor identities will be collected as necessary to improve synthesis understanding. The inventors also plan to conduct broader materials searches for other potentially interesting MIT material families along with their synthesis information for further analysis.

### 2 Machine Learning-Assisted Concept Exploration

The inventors successfully built a binary classification model that rapidly determines whether a given composition and structure is likely to exhibit a metal-insulator transition. The inventors also identified a key material descriptor via this model. The inventors advanced this model and applied it to lacunar spinel compounds. The inventors also initiated the implementation and organization of relevant code, data handling, and ML pipeline infrastructure necessary for successful operation of the CVAE model.

### M2.1 Classification ML Model Created for Predicting Candidate Materials Families

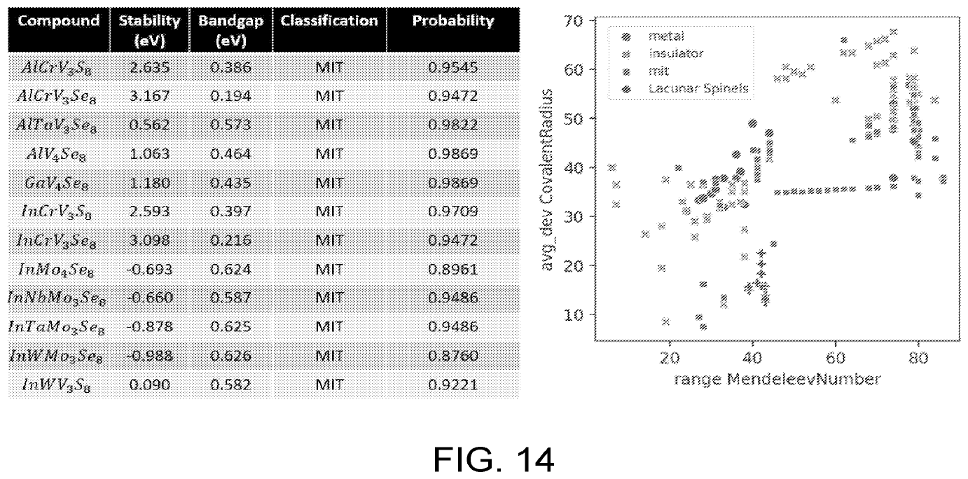

Built upon results from M1.2 and M1.3, the inventors advanced a novel machine-learning binary classification model to ascertain whether a given composition and structure would exhibit a metal-insulator transition. This classifier is key to the success of the Concept Exploration tasks and will allow us to screen many published high-throughput DFT databases for previously identified MIT compounds and materials families. In this period, the inventors utilized the classifier to evaluate lacunar spinel compounds predicted on the Pareto front from the multi-objective LVGP (see Sec. 3 and 5) and focused on understanding the physical origin of the important features. All compounds identified by the LVGP model were also identified as MIT compounds (as shown in FIG. 14), indicating the power of the LVGP method for achieving featureless learning.

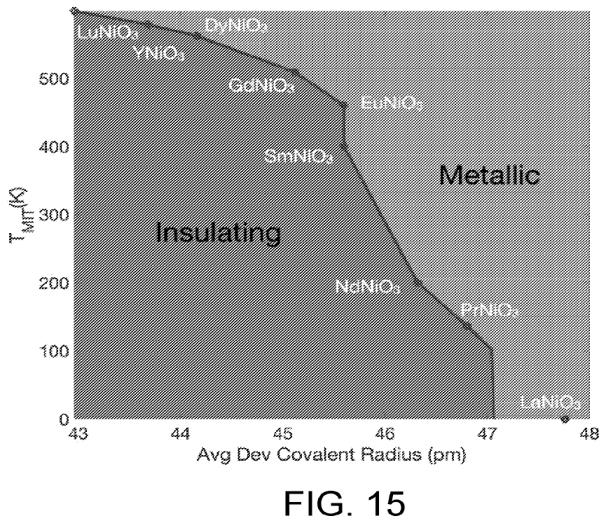

First, the inventors identified the Average Deviation of the Covalent Radius (ADCR) identified previously as a key feature giving the MIT classifier high performance. The inventors examined the role of this feature on MITs in the rare-earth perovskite RNiO3 family (R=rare earth element). This family exhibits bandwidth-controlled MIT transitions that arise from changes in the NiO6 octahedral rotation amplitude. The tendency of a material in the perovskite family to exhibit octahedral rotations is described using the Goldschmidt tolerance factor t=(rR+rO)/[2^{1/2}(rNi+rO)^{1/2}] comprising the ionic radii of the R and Ni cations and oxygen anions. For lower values of t, the transition metal-oxygen octahedra rotate, making it more difficult for electrons to hop and favoring a MIT. Thus, a lower t value usually leads to a higher MIT temperature, while a higher t would suppress the MIT behavior altogether (e.g., LaNiO3 is metallic). Here, the inventors identified the ADCR is linearly correlated to the tolerance factor t, and the inventors show the dependence of the MIT transition temperature TMIT as a function of ADCR, as shown in FIG. 15.

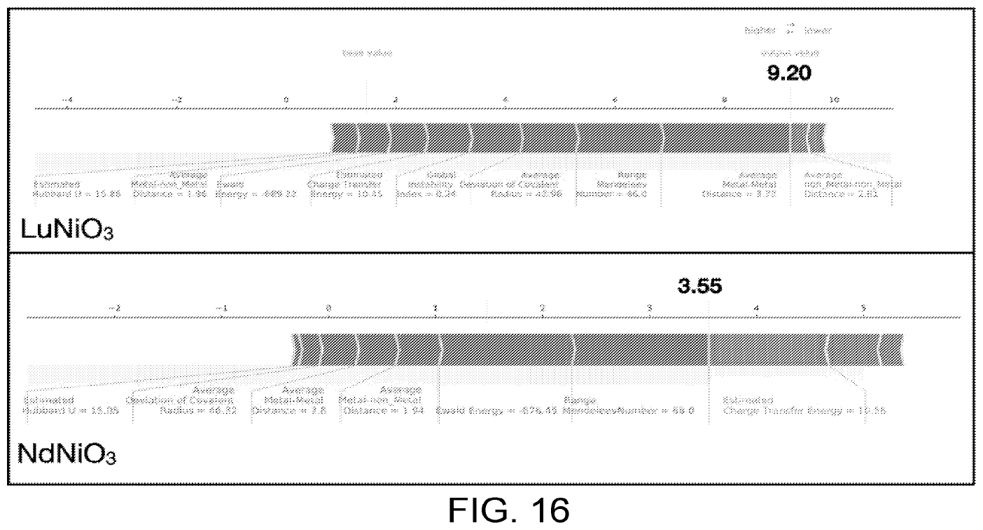

The model captures this physics through SHAP values, as shown in FIG. 16. As shown in FIG. 16, color indicates whether the feature taking the particular value was providing evidence for (fuchsia) or against (blue) a prediction of having a MIT. Bar size represents the SHAP value. SHAP values from all features sum up to the log-odds of a positive MIT prediction. The base value is the log-odds expected based on the average proportion of MITs in the dataset. The classifier predicts with higher confidence that LuNiO₃ is an MIT material than NdNiO₃, with a stronger role from the ADCR. NdNiO₃ (NNO) exhibits one of the highest ADCR values in the nickelate family and the lowest TMIT. This positions it close to the metallic phase and is identified by the classifier model as having a log-odds probability of 3.55. In contrast, LuNiO₃ is identified as having a 9.2 probability. The ADCR for NNO less strongly pushes the prediction towards a MIT classification compared to LuNiO₃, which the model shows is strongly in favor, clearly capturing the experimental physical trend. The ADCR is thus likely similar to a generalized tolerance factor, irrespective of the materials family studied.

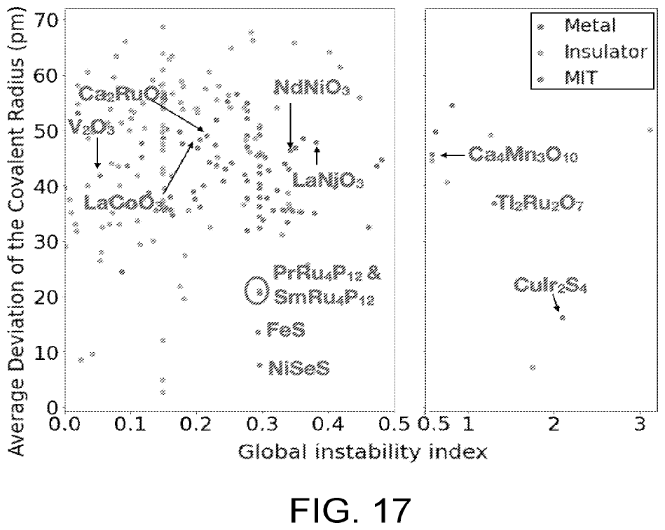

A second feature the inventors identified to be important is the Global Instability Index (GII), which describes the deviation of bond lengths in a particular material from ideal bond lengths based on bond valence analyses. If the GII is too large or too small, the structure is likely to be less stable and microscopic mechanisms such as those responsible for an MIT may become less likely to occur. Indeed, the inventors find that most MIT materials are concentrated around a GII of 0.3 (see FIG. 17). As shown in FIG. 17, most MIT compounds tend to have an intermediate ADCR and GIL. Notable exceptions with low ADCR are FeS, NiSeS, CuIr₂S₄. The compound with the lowest GII is V₂O₃, which is likely responsible for its much larger structural response to the MIT. GII values are obtained based on the high temperature structure for MIT compounds.

### M2.2 CVAE Deep Learning for Generating Synthesis Recipes

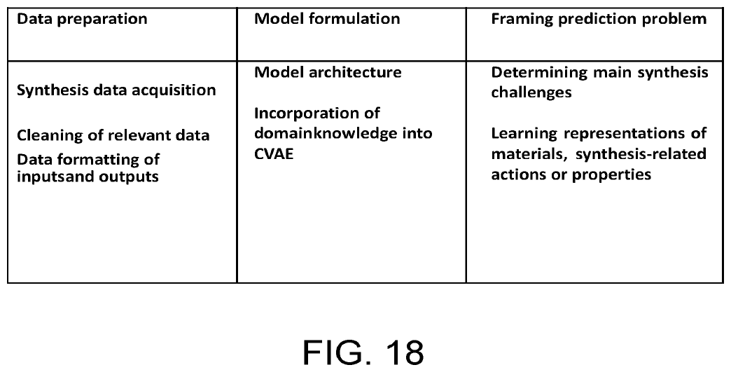

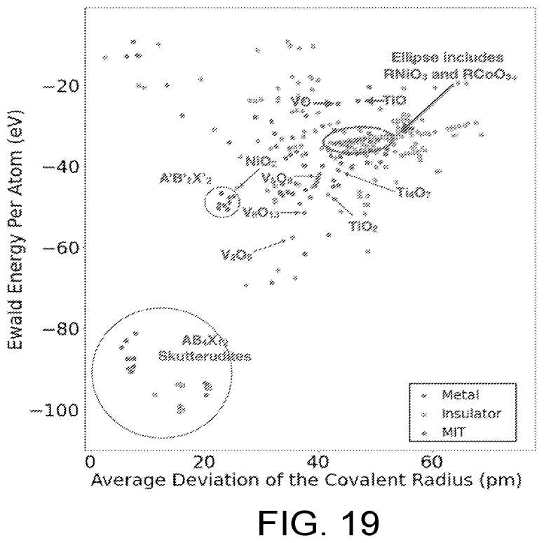

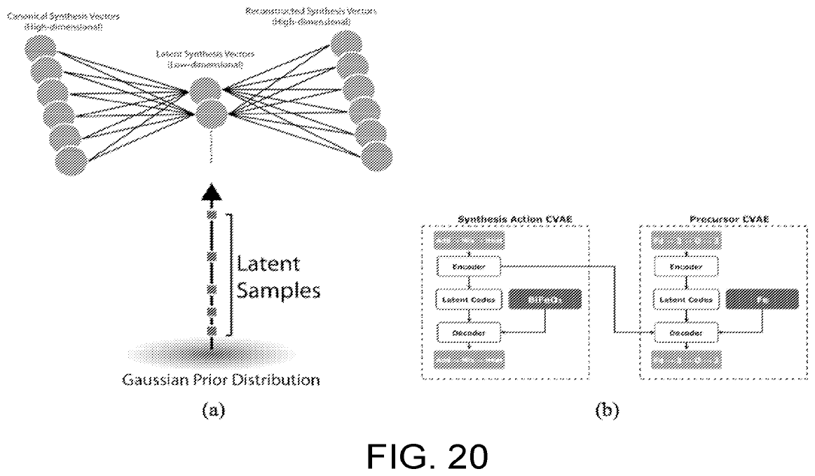

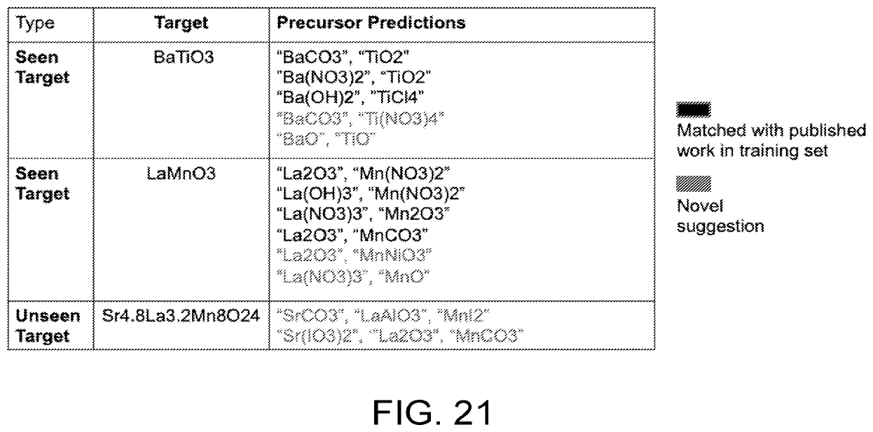

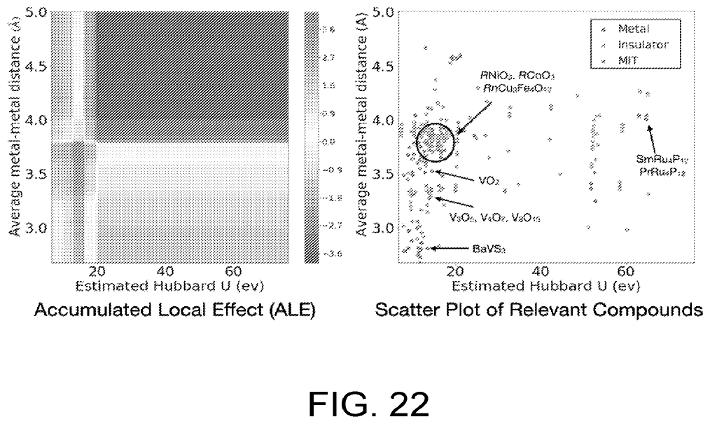

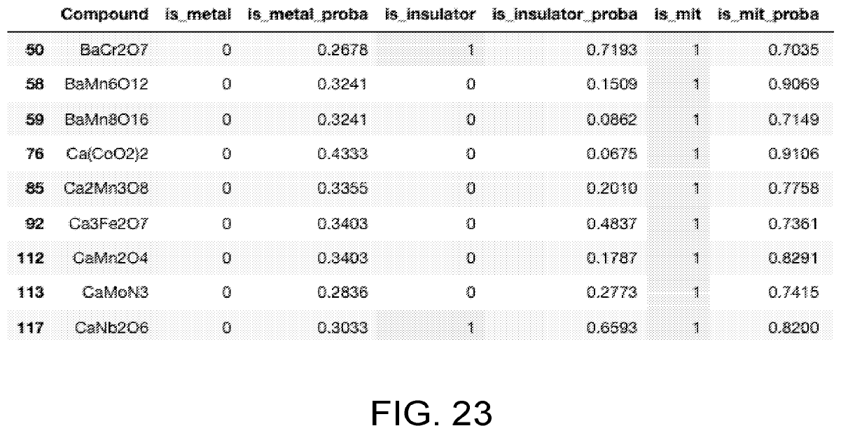

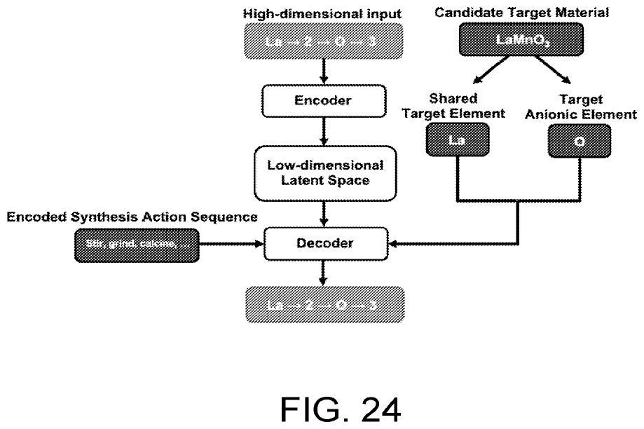

The relevant code, data handling, and ML pipeline infrastructure necessary for successful operation of the CVAE model were begun to be implemented and organized. A summary of current ongoing tasks involving the CVAE model are in a table as shown in FIG. 18. Data preparation involves the acquisition, cleaning, and formatting of relevant synthesis data as described in M1.3. This step additionally involves thinking about the proper formatting of inputs and outputs to the CVAE model, such as converting textual synthesis information into data formats that can be read by an ML model including word embeddings, one-hot encoded vectors, and other representative data formats. Model formulation includes designing the proper ML model architecture to accept the data inputs as well as incorporating domain knowledge from the MIT material literature into the model in order to improve prediction. Framing of the proper prediction problem is also a current ongoing effort, as it is crucial to determine the main synthesis challenges of the metal-insulator material literature to better understand the ways in which the CVAE model could aid understanding of material synthesis in a particular area. This additionally involves thinking about the way in which the learned representations of MIT materials and their synthesis-related actions or properties may be incorporated into the CVAE model.

In the next period, the inventors plan to use the classifier to search for additional MIT material families and further understanding the physical origin of important features by applying the aforementioned analysis to the RCoO₃ and RCu₃Fe₄O₁₂ materials families. At present, a preprint of this work has been posted on arXiv.org. The MIT classifiers were presented in the webinar titled “Machine Learning for Scarce Material Classes: Understanding and Predicting Metal-Insulator Transition Compounds” at the AI for Materials: From Discovery to Production Meeting on Oct. 6, 2020. The MIT classifiers were also packaged and made publicly available. They are easily accessible via an interactive Jupyter notebook hosted by Binder. Anyone can upload a structure file in CIF format and make their own prediction using the interactive Jupyter notebook. Since this notebook is hosted in a Docker containerized environment, anyone interested in making a classification on their own material can execute the script immediately in their web browser without installing any dependencies. This aspect greatly improves the usability of the code, especially by non-computational researchers. The complete workflow behind the ML models is described in the project's GitHub page with some sub-functions also demonstrated in an interactive Jupyter notebook.

### 3 DFT-Based High Fidelity Evaluations

The inventors successfully implemented and validated DFT+Hubbard U calculations to accurately capture the complex physical mechanisms underlying metal-insulator transitions such as the Jahn-Teller-type distortions. The inventors used the methods to study the MIT mechanisms in the lacunar spinel compositions on the Pareto front of band gap and synthesizability.

### M3.1 DFT Simulations Completed for Assessing Mechanism and Responses of at Least One MITs Family

The inventors used the high-fidelity DFT methods to accurately study the MIT mechanisms in the lacunar spinel compositions identified on the Pareto front (see details in M4.1) as defined by band gap and synthesizability (decomposition energies). The inventors use DFT simulations to examine these properties and the microscopic mechanisms of the MIT, focusing on the Jahn-Teller active phonon involved in the transition. It was found that most Pareto-front compositions consist of two different cations with 75% of the optimized materials being selenides (see FIG. 14). GaV4Se₈ is the only Pareto front compound previously synthesized and verified to exhibit resistive-switching behavior under an applied electric pulse. All compounds exhibit R3m symmetry and are dynamically stable in their ground state. The phonon frequencies of the selenides are lower than those of the sulfides. All of the designed lacunar spinels also exhibit semiconducting gaps with semi-local exchange-correlation and static Coulomb interactions with the DFT simulations and exhibit nonzero electric polarizations. The inventors also identified that compositions with larger band gaps tend to have lower thermodynamic stability.

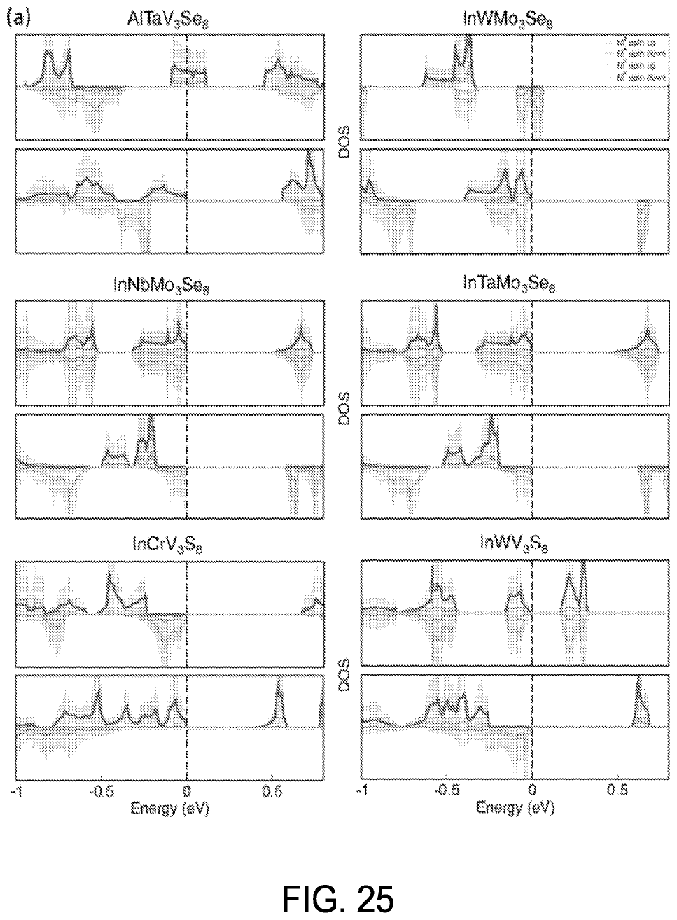

FIG. 25 shows DFT-simulated electronic properties of selected lacunar spinel compositions at the Pareto front according to certain embodiments of the present invention. The lower panel of each composition shows the ground state electronic structure and the upper panel shows the DOS of the metastable phase after the Jahn-Teller distortion. Both panels are normalized and span a range of 15 states per formula unit for each spin channel (vertical axis). AlTaV₃Se₈, InWMo₃Se₈ exhibit metal-insulator transitions whereas the other compounds show semiconductor-to-insulator transitions.

Although the ground states of the lacunar spinels are all semiconducting, the inventors found two different electronic transitions: the expected (Type I) metal-to-insulator transition and an unexpected (Type II) semiconductor-to-insulator transition (SIT). FIG. 25 shows the changes to the electronic structure for the MIT lacunar spinels AlTaV₃Se₈ and InWMo₃Se₈ with the insulating state (lower panel) always lower in energy than the metastable metallic phase (upper panel) after the Jahn-Teller-type distortion. The projected density-of-states (pDOS) of these compounds show that the metallic state in the Type I transition arises from cluster distortion-triggered orbital ordering and occupancy changes. However, the metallic states are different owing to the chemistry of the metals. The inventors also find that the basal metal atoms play a more decisive role near the Fermi level with minor contribution from the apical metal site. The remaining lacunar spinels as shown in FIG. 25, InNbMo₃Se₈, InTaMo₃Se₈, InCrV₃S₈, and InWV₃S₈, exhibit a Type II transition. The lower and upper panel show their ground and metastable state pDOS, respectively. Interestingly, some compounds undergo singlet formation and transform into a nonmagnetic phase (e.g., InNbMo₃Se₈) while others remain ferromagnetic after the cluster distortion (e.g., InCrV3S8) owing to competition between spin-pairing and magnetic interactions.

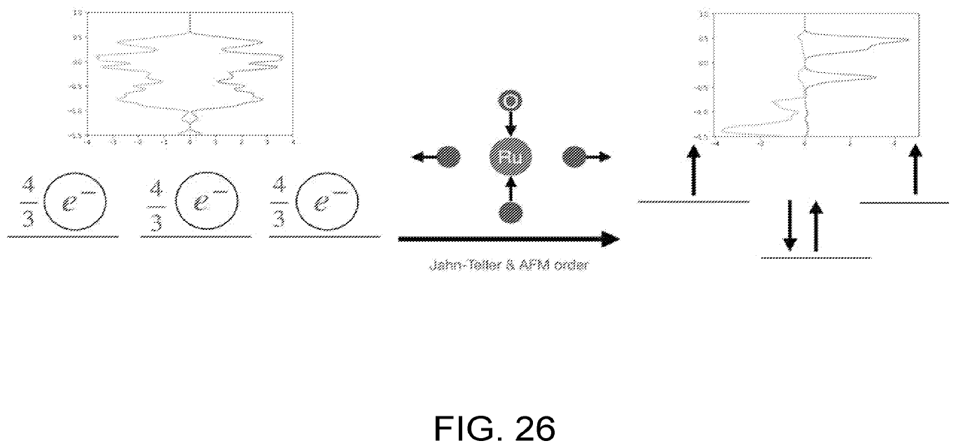

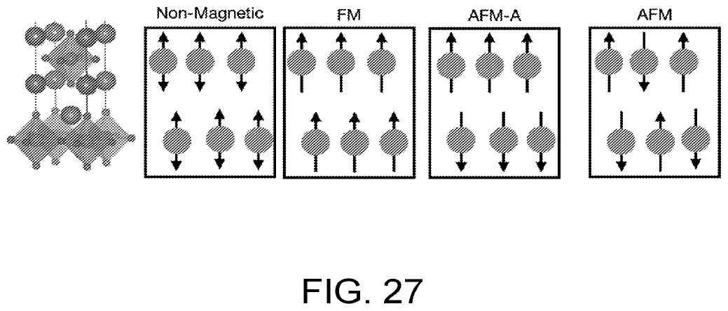

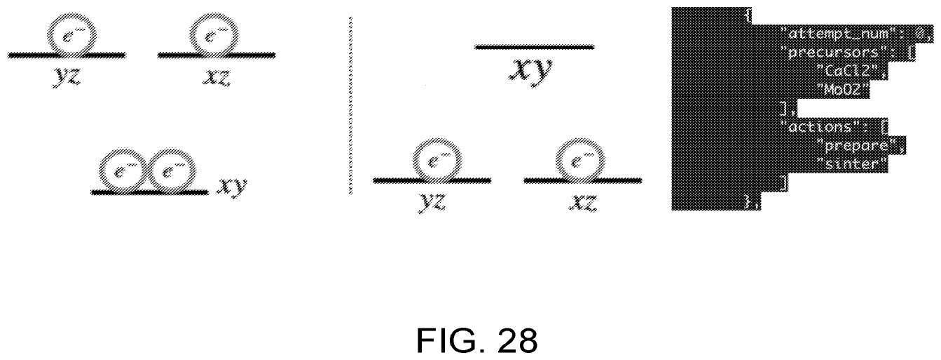

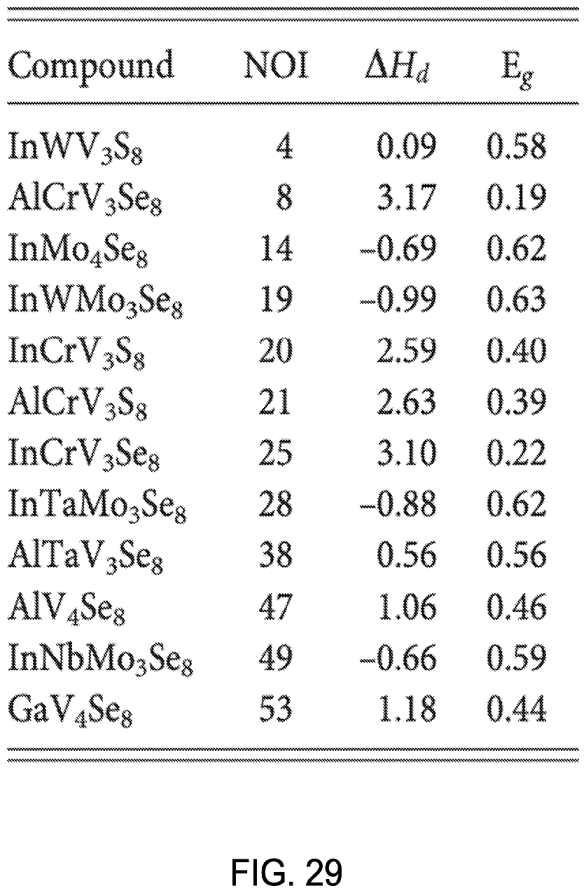

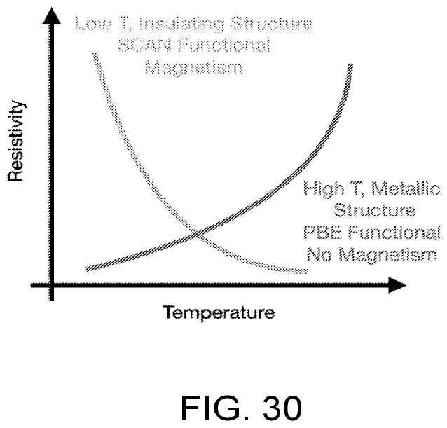

After identifying that the high-fidelity DFT calculations for the Ruddlelsden-Popper (RP, An+1BnO3n+1) perovskite structures will utilize the SCAN metaGGA functional, the inventors worked to optimize the numerical solvers early in this period. The inventors also decided to focus on the n=1 family: A=Ca, La, Sr, Ba, and B=Ti, V, Cr, Mn, Fe, Co, Ni, Cu, Zr, Nb, Mo, Tc, Ru, Rh, Pd, Ag. For each of these compounds, the inventors simulated 4 possible magnetic states. This work involved two main steps calculation: (1) a low-resolution calculation, similar to those performed by Materials Project. This then feeds into (2) a high-fidelity calculation that allows us to fully identify the mechanism for an MIT—if such a mechanism exists. The inventors have performed all low-resolution calculation for these compounds and are in the process of obtaining and analyzing the higher resolution calculations for these compounds (see FIG. 26). The inventors will use a combination of these results, the MOBO framework and the online classifier to further down select to the most important MIT to suggest for experimental growth.

The future plan of DFT simulation includes the following: complete DFT calculations on the RP family of materials and initiate DFT calculations aimed at understanding nonstoichiomietry (x) and oxygen vacancies δ for the La1−xCoO3−δ family. The inventors will then combine the DFT-optimal parameters with the LVGP based adaptive optimization for MIT materials design.

### 4 LVGP Machine Learning

This task started where the inventors did an initial design of experiments and completed the LVGP modeling for the lacunar spinel family. The inventors are also extending LVGPs to multi-response situations and to large datasets.

### M4.1 Initial DoE of MITs Training Data Collected for LVGP Modeling

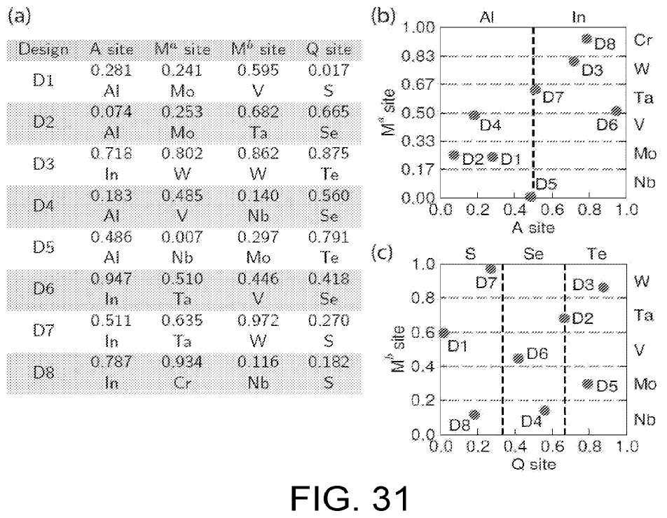

The inventors start the MIT-materials AOE for the lacunar spinel family through an initial design of experiment (DoE) consisting of four experimentally known compounds within the family (i.e., GaMo₄S₈, GaV₄S₈, GaNb₄Se₈, and GaTa₄Se₈) and eight new compositions generated by discretized Latin Hypercube Design (LHD) shown in FIG. 31. A four-dimensional LHD of size eight is generated, where each dimension corresponds to a crystal site (e.g. A, Ma, etc.). Since the four known compounds are all gallium-based, the inventors only consider Al and In for the A site design. As shown in FIGS. 31(b) and (c), each dimension is evenly divided into a number of grids, each grid represents one candidate elemental composition at that crystal site. For instance, the Q site is divided into three grids because there are three candidate elements (S, Se, Te) on that site. The designed composition could then be determined using the grid-composition correspondence. For example, Design ID Number 1 (D1) resides in the grid corresponding to {Al, Mo, V, S}; therefore, its composition is AlMoV3S8. This procedure ensures a variety of elemental combinations within the initial DoE set, where each candidate element will appear at least once, so that the model has knowledge about different elemental contributions to the design objectives. The simulation data is then used for LVGP based Bayesian Optimization under M5.1.

### M4.2 Multi-Functional Response LVGP Developed

Multi response Gaussian Processes (MRGPs) are natural extensions of GPs that allow to model RFs with multiple responses. For an Random Field (RF) with q outputs y=[y₁, . . . , yq]^{T} and the field (e.g., spatial or temporal) inputs x=[x₁, . . . , xd]^{T}, the best linear unbiased estimator representation of an MRGP with constant means reads as:

$$y ∼ Nq ( β , c ( x , {x}^{′} ) ) ,$$

where Nq represents a q-dimensional Gaussian process, β=[(β1, . . . , βq]T is the vector of responses' means, and c(x,x′) is a parametric function that measures the covariance between the responses at x and x′. One common choice for (x,′) is:

$$c ( x , {x}^{′} ) = \sum ⊗ exp \{ {\sum }_{i = 1}^{d} - {10}^{{ω}_{i}} {( {x}_{i} - {x}_{i}^{′} )}^{2} \} = \sum ⊗ r ( x , {x}^{′} )$$

where Σ is a q×q symmetric positive definite matrix that captures the marginal variances and the covariances between the outputs, d is the dimensionality of the field, ω=[ω1, . . . , ωd]^{T} are the so-called roughness or scale parameters that control the smoothness of the RF, and ⊗ is the kronecker product. Note that the dimension of β and Σ depends on q while that of ω depends on d. The parameters β, Σ, and ω enable an MRGP to model a wide range of random processes:
   The mean values of the responses over the entire input space are governed by β.
   The general correlation between the responses (i.e., yi and, i≠j) over the input space is captured by the off-diagonal elements of Σ.
   The variations around the mean for each of the responses are controlled by the diagonal elements of Σ.
   The smooth/rapid changes of the responses across the input space are controlled by ω. In case some experimental data are available, all the hyperparameters of an MRGP model can be estimated via, e.g., the maximum likelihood method. The inventors plan to extend this strategy for GP models with continuous inputs to mixed variable multi-response LVGP.

### M4.3 LVGPs for Large Dimensions and Datasets Developed

Through the concept exploration and adaptive discovery pipeline, the inventors expect to discover and investigate an increasing number of MIT materials and families. The inventors also expect an increasing number of dimensions of the atomic structure-composition variable spaces. The goals for this milestone are to efficiently scale LVGPs to an increasing number of observations, and to develop a mechanism for handling a large number of dimensions. GPs in general do not scale well with the number of training observations. To see this, consider training a GP on N training points X_{N}=[x^{T}, . . . , x^{T}]^{T}, and their corresponding observations y_{N}=[y1, . . . , y_{N}]^{T}. The log-likelihood function for the GP model is given by

$$log p ( {y}_{N} \vert {X}_{N} θ ) ∝ - log \vert {K}_{NN} \vert - {y}_{N}^{T} {K}_{NN}^{- 1} {y}_{N} ,$$

where θ represent the GP hyperparameters, and KNN is the N×N covariance matrix of the observations. The computational expensive terms in the log-likelihood are the matrix solve K⁻¹ yN, and the log-determinant log|KNN|. Standard approaches use Cholesky decomposition, which requires (N³) computations and O(N²) storage. This scaling problem naturally affects LVGPs as well. Scalable GP approaches usually introduce approximations to the GP model. The idea common to all of these approaches is to compute the Cholesky decomposition of the covariance matrix on observations at a much smaller number of points. These approaches, however, are designed for quantitative inputs. Some modifications are needed when applying to qualitative inputs and LVGPs in particular.

The inventors have been working on extending the stochastic variational inference (SVI) approach to LVGPs. Let (⋅) denote the GP model, and let fN=[(x1), . . . , f(xN)]^{T} denote the function values at training points XN. SVI first augments the method with a set of M<<N inducing points \tilde{X}_{M}=[\tilde{x}^{T}, . . . , \tilde{x}^{T}]^{T}, and their corresponding function values u=[f(\tilde{x}1), . . . , f(\tilde{x}M)]^{T}. SVI approximates the joint posterior distribution (fN, u|yN) with the variational distribution (fN, u|yN):

$$p ( {f}_{N} , u \vert {y}_{N} ) \approx q ( {f}_{N} , u \vert {y}_{N} ) = p ( {f}_{N} \vert {y}_{N} ) q ( u ) ∼ 𝒩 ( m , S ) ,$$

where m∈ [symbol] , and S∈ [symbol] are learnable variational parameters. These parameters, along with the GP hyperparameters and \tilde{X}M are found by maximizing the evidence lower bound:

$$ℒ = {\sum }_{i = 1}^{N} {𝔼}_{q ( f ( {x}_{i} ) )} [ log p ( {y}_{i} \vert f ( {x}_{i} ) ) ] - KL [ q ( u ) ‖ p ( u ) ] ,$$

where q(f(xᵢ))=∫p(f(xᵢ)|u)q(u)du, and KL[⋅∥⋅] is the Kullback-Leibler divergence between two probability distributions.

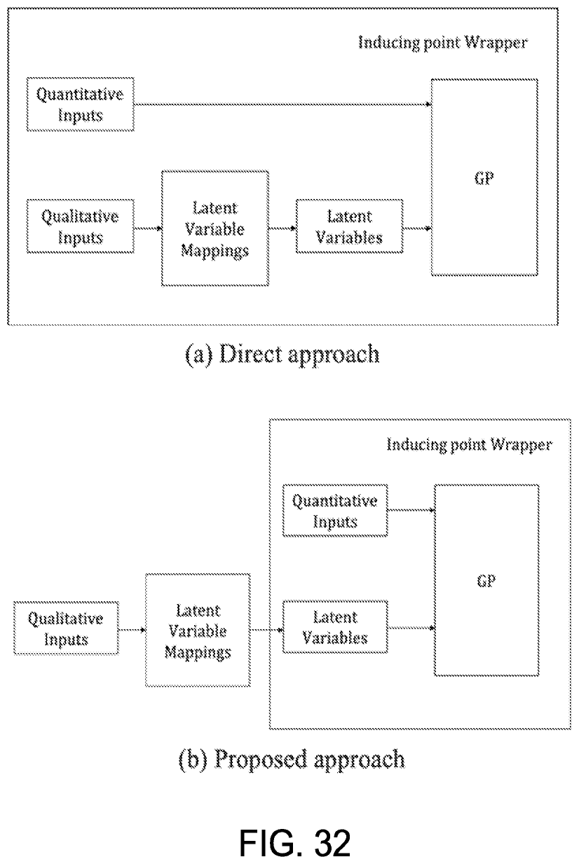

This method requires (M³) computations, and hence can be scaled to large datasets. Since its objective function involves a summation over the data points, SVI can be used in conjunction with stochastic gradient optimization techniques for memory savings. An issue in applying SVI to LVGPs is that the inducing points \tilde{X}M are now defined over a mixed qualitative-quantitative space and cannot be optimized via gradient based methods. Heuristic methods may be needed to optimize over this space. They might potentially increase the computational costs. Moreover, convergence is not necessarily guaranteed with heuristics. Instead, the inventors proposed to define the inducing points in the joint space of the quantitative inputs and latent variables as opposed to the mixed qualitative-quantitative space, as shown in FIG. 32. With this modification, the model can now be optimized via gradient based techniques. The inventors refer to this model as the LVGP-SVI model.

Instead of approximating the GP model, an alternative method to reduce the computational costs is to use numerical linear algebra approximations in lieu of the Cholesky decomposition. The black box matrix-matrix multiplication (BBMM) method uses preconditioned conjugate gradients for computing the matrix solve

$${{K}_{NN}}^{- 1} yN ,$$

and stochastic trace estimators for an unbiased but scalable estimate of the log-determinant and its gradients with respect to the GP hyperparameters θ. BBMM reduces the computations from (N³) to O(N²), thus improving scalability while maintaining exact inference. It makes no assumptions about the kernel or the inputs, and hence can be directly used for scaling LVGPs. The inventors will refer to this model as the LVGP-BBMM model.

The inventors now compare the prediction quality and scalability of the two approaches: LVGP-BBMM and LVGP-SVI. The inventors consider a dataset of 2030 ternary oxide materials (AxByOz) extracted from the Open Quantum Materials Database (OQMD). The goal is to predict the formation energy and the stability of the materials from only the compositional information (A,B,x,y,z). Element A belongs to either the first two columns of the periodic table or the lanthanide rows, and there are 25 such elements in the dataset. Element B is a transition metal, and there are 22 candidates in the dataset. While the moderate size of the dataset is by no means prohibitive for training a LVGP model with no approximations, it is still memory intensive to train one. The inventors use single-precision floating points when training to ease the memory requirements. A challenge in this dataset is the large number of levels for the two qualitative variables. To deal with the large number of latent variables in the LVGP models, the inventors add a weight-decay regularization penalty for the latent variables. The penalty is to be tuned by cross-validation (CV).

In addition to the LVGP models, the inventors also train a neural network and a random forest model for comparison. The inventors use Bayesian optimization to tune the different models using 10-fold CV.

The inventors compare the different models using replicated 10-fold CV estimate of the relative root mean squared error (RRMSE) in predicting the two quantities. The inventors also compare the different models based on their training time on the entire dataset. The inventors measure the training time across 20 different runs and report the mean and standard deviation. Note that the training time does not include the time for tuning the models via Bayesian optimization.

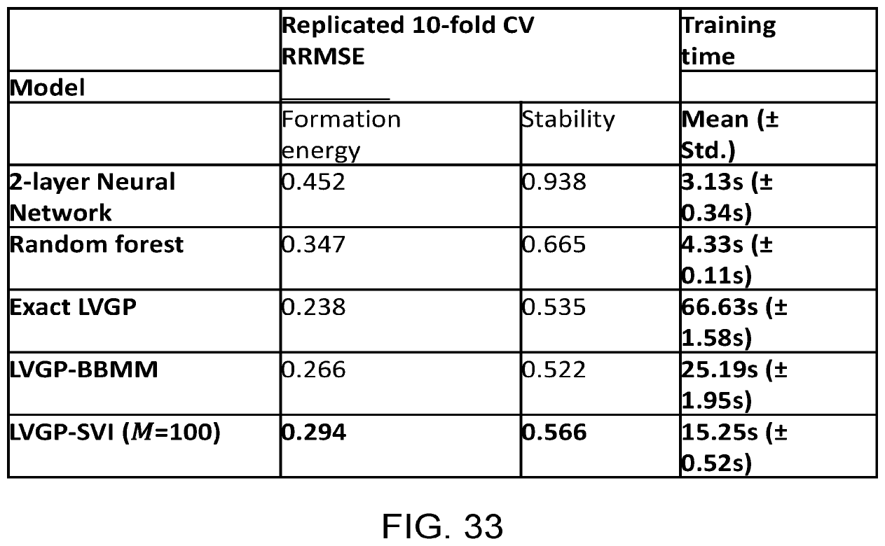

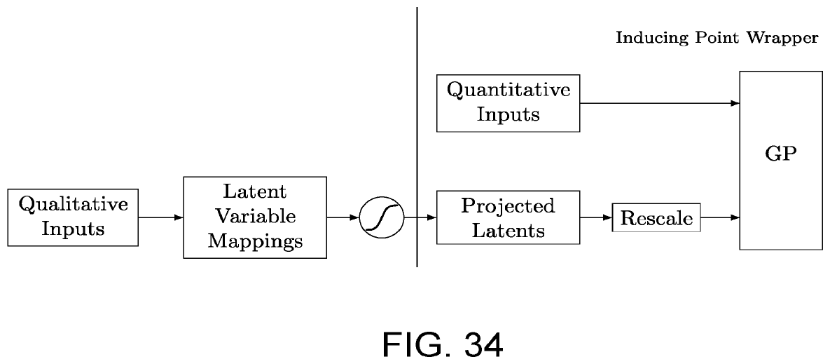

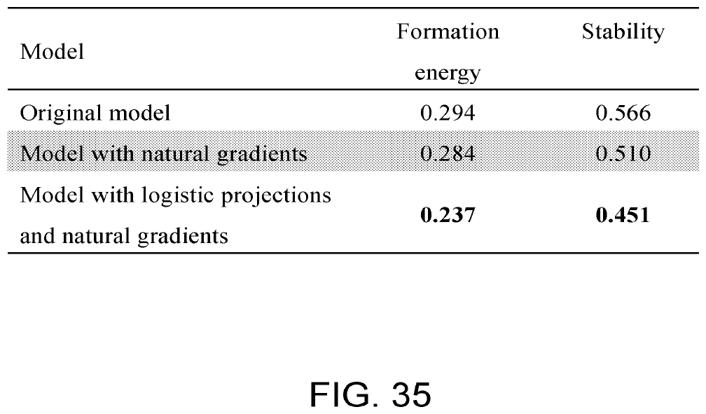

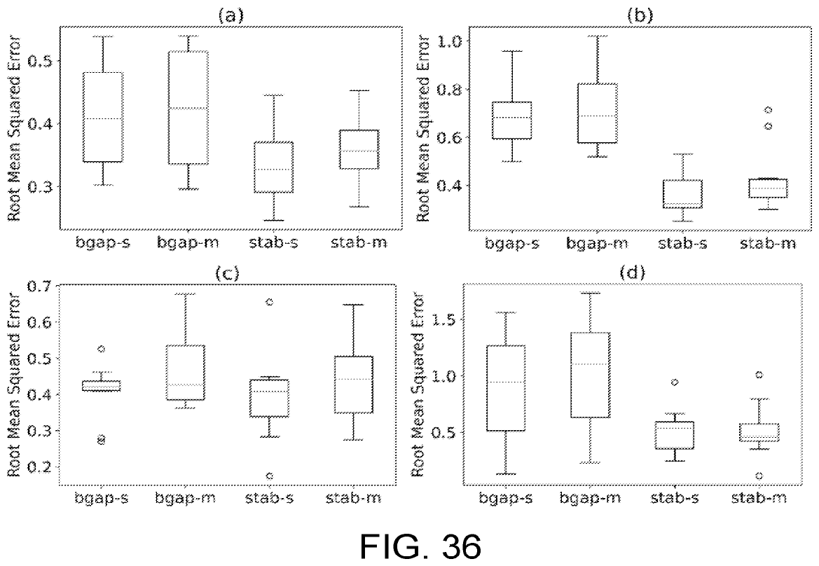

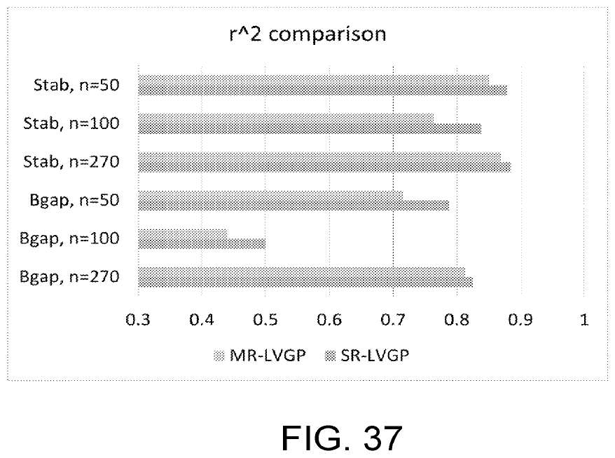

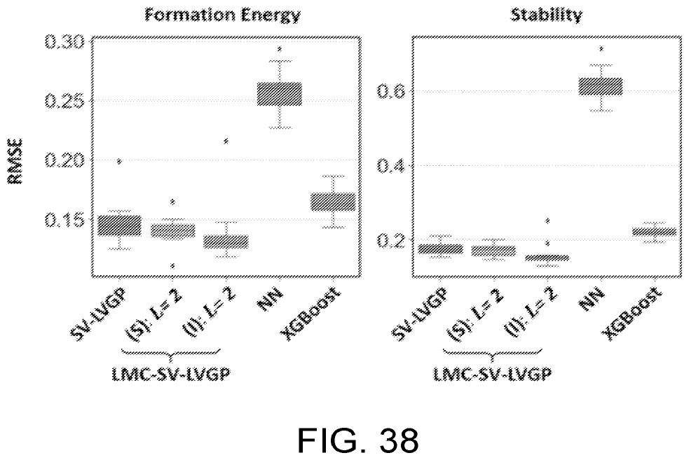

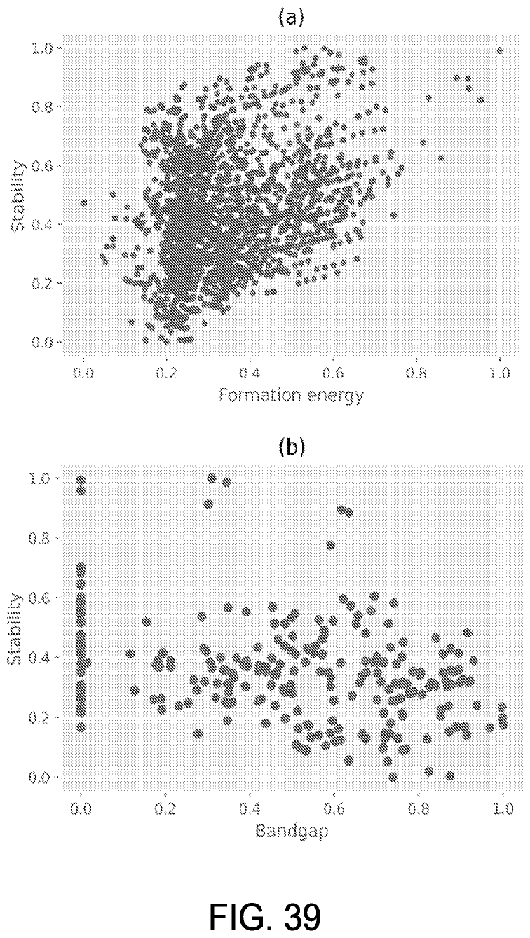

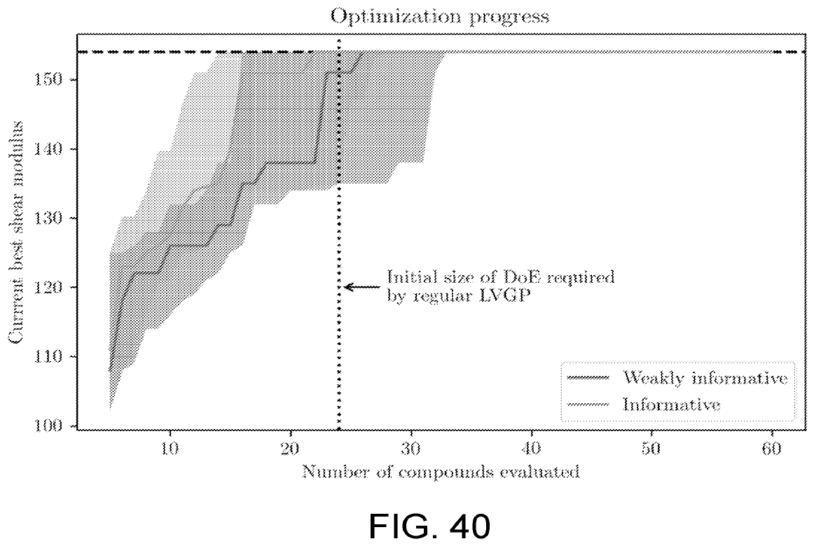

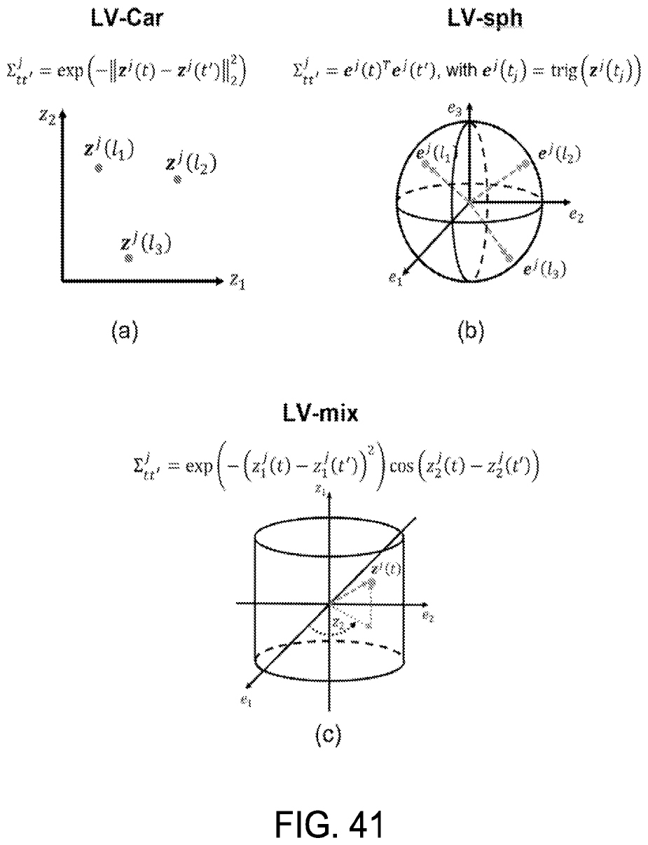

The results are shown in the table as shown in FIG. 33. The LVGP models have much better predictive performance than the neural network and random forest models. The exact LVGP model has slightly better performance than LVGP-BBMM for predicting formation energy. An unexpected result is the exact LVGP performing slightly worse than the LVGP-BBMM for predicting stability. This could be due to the round-off errors introduced by the single-precision floating points. The LVGPBBMM model is more than twice as fast as the exact LVGP model. The LVGP-SVI model with 100 inducing points does only slightly worse than the LVGP models with exact inference, while being faster than both methods. Increasing the number of inducing points could potentially increase the predictive performance, while taking slightly longer to train.

These initial results show that the scalability of the LVGPs can be potentially improved with some performance tradeoff. These results also show that LVGPs can potentially handle qualitative variables with a large number of levels by appropriately regularizing the associated latent variables. The inventors plan to rigorously test these approaches on a diverse variety of medium and large datasets to detect and diagnose potential issues.

An attractive feature of GPs is the availability of principled uncertainty estimates. Uncertainty quantification is important for many applications such as Bayesian optimization and active learning, where good uncertainty estimates are needed to drive effective exploration. The inventors plan to run experiments to evaluate the impact of the two approximations on uncertainty quantification. This can be a concern for the LVGP-SVI model which introduces approximations to LVGP inference, and with the inducing points being defined in the joint space of the quantitative inputs and latent variables. For the LVGP-SVI model, there is also the task of choosing the number of inducing points to balance the performance-computation trade-off. To this end, the inventors plan to empirically analyze and quantify this tradeoff across different sets of problems with varying data sizes, and then develop a mechanism to identify a good number of inducing points for any given problem. The inventors also plan to compare LVGP models with alternative machine learning models in the large data situation, and potentially combine elements of both types of models to achieve better scalability.

### 5 Mixed-Variable LVGP-Based Bayesian Optimization

The inventors advanced the mixed-variable Latent Variable Gaussian Process (LVGP) method for uncertainty quantification within a Bayesian representation and employed it in a multicriteria Bayesian Optimization (BO) framework for mixed-integer problems. The inventors concluded this task using the method to find novel lacunar spinels that exhibit high thermodynamic stabilities and large resistivity-switching ratios.

### M5.1 Multicriteria LVGP-Based Bayesian Optimization Developed (BO)

The complex lacunar spinel family [symbol] ^{a}M^{b}Q₈ with trivalent main group A∈{Al, Ga, In}, transition metals M^{a}∈{V, Nb, Ta, Cr, Mo, W}, M^{b}∈{V, Nb, Ta, Mo, W}, and chalcogenide Q∈{S, Se, Te} ions demonstrate the complexity active in MIT materials design. In pursuit of novel MIT materials with superior performance, the inventors specifically seek lacunar spinels that exhibit high thermodynamic stabilities (ΔHd) and large resistivity-switching ratios, which the inventors formulate as two design objectives for the materials discovery task. Materials with larger ΔHd are expected to be more synthesizable and stable during operation, making it a useful filter to prioritize compounds for subsequent theoretical analysis and synthetic processing. The second design objective is the ground state band gap (Eg). The inventors use it as a proxy for the resistivity-switching ratio since Eg is positively correlated with the resistivity change between different electronic states. With four categorical variables (270 compounds) and two design objectives, this problem presents an excellent testcase for the Multi-Objective BO (MOBO) using LVGP.

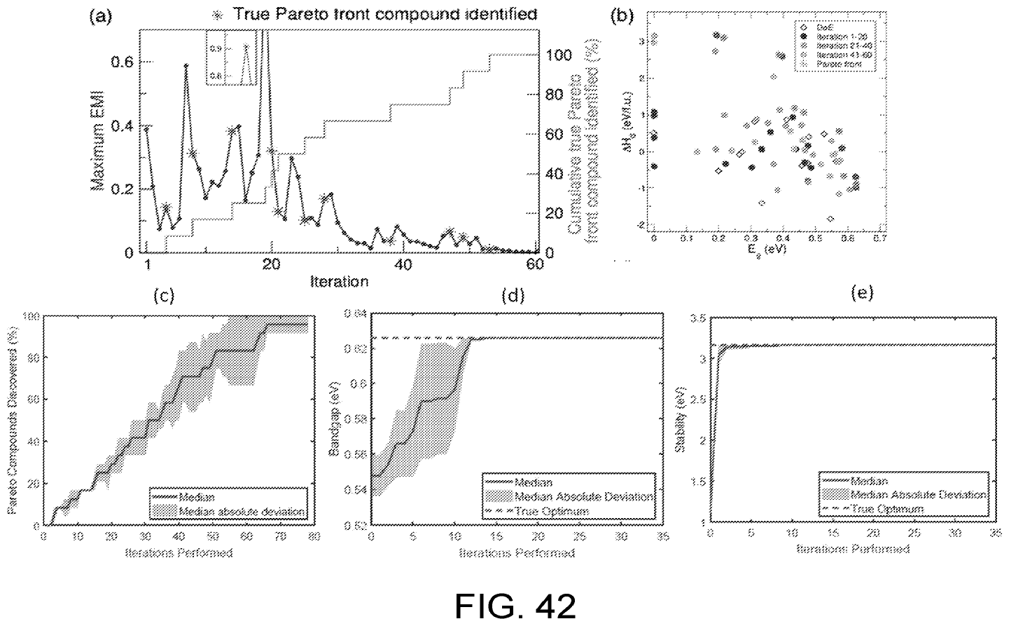

Starting with repository of 12 compounds selected via Design of experiment, the inventors performed several iterations of MOBO. Each iteration uses two independent LVGP model to predict the two objectives and associated uncertainty for spinels that are not present in the repository. This information is used to compute the Expected maximin improvement (EMI) and the composition with largest EMI is simulated using DFT. The inventors notice that all 12 Pareto front compositions are identified within 60 iterations, as shown in FIG. 42(a). The value of EMI is consistently zero after all Pareto front compositions are identified, indicating that there is no other composition that could offer improvement. Since the two objectives cannot be optimized simultaneously, these 12 compounds on Pareto front represent the optimal tradeoff between them. FIG. 42(b) shows the history of composition explored for the first 60 iterations. The initial DoE sets are relatively scarcely distributed away from the true Pareto front, yet the model explores regions far from that covered by the DoE sets and is able to identify 75% of Pareto-front compositions within the first 40 iterations.

After repeating MOBO 10 times, each time with a different DoE, the inventors noticed that the majority of compounds on the Pareto front were discovered with 90 iterations i.e. less than 30% exploration of design space, as shown in FIG. 42(c). Although designing materials under a single criterion is more efficient as shown in FIGS. 42(d) and (e), such efforts may not meet the requirements of deployment. For lacunar spinels investigated here, maximizing Eg exclusively leads to an unstable composition while maximizing ΔHd exclusively leads to a composition with a small bandgap. In contrast, MOBO identifies the Pareto front to delineate the trade-off between materials' properties and allows the designer to choose compositions for detailed study. In this context, the need to perform more iterations of MOBO is justified. Indeed, it is typically not the sole goal to find all Pareto-front designs, but rather to identify the best candidates within a limited research budget.

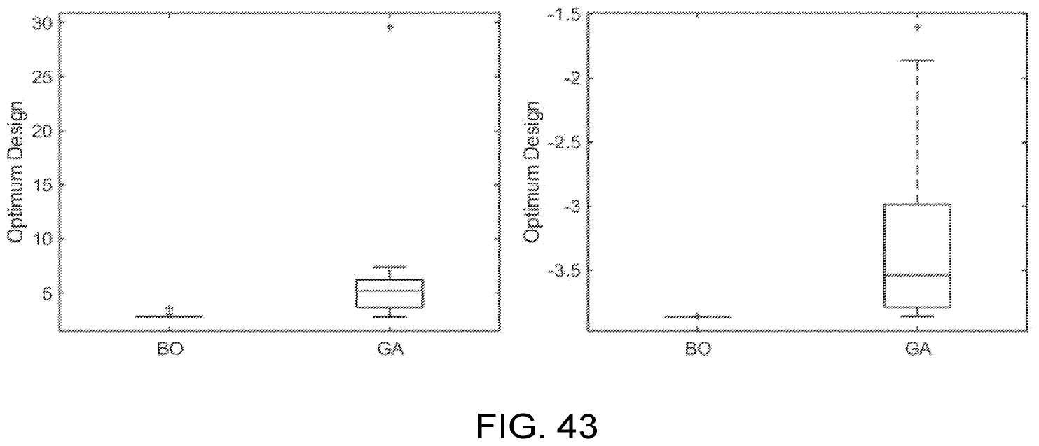

FIG. 43 shows comparison of best design identified by Bayesian Optimization (BO) and Genetic Algorithm (GA) after 25 objective function evaluations of 2D Branin function (left) and 3D Hartmann function (right).

The inventors use DFT simulations to examine the properties of the identified Pareto front compositions, focusing on ΔHd, Eg and the Jahn-Teller active phonon frequency (vJT) listed in the following table. Although the ground states of these materials are all semiconducting, the inventors find two different electronic transitions upon traversing the ideal Transition Metal Cluster geometry: the expected metal-to-insulator transition (MIT) and an unexpected semiconductor-to-insulator transition (SIT). To demonstrate the further integration of submodules, the inventors also investigated the predictions that the ML classification model (M2.1) made for the Pareto compositions. Note that the MIT classifier has not seen these compounds before. As seen by the classification and its corresponding probability listed in the following table, all 12 compositions are predicted to be MIT with great confidence. This result is encouraging as it presents strong justification for experimental groups in academia and industry to pursue testing of these novel compounds.

TABLE
analysis of Pareto front lacunar spinels

| Pareto | ΔH_{d} | E_{g} | v_{JT} | Type of | Classifi- | Proba- |
|---|---|---|---|---|---|---|
| Compound | (eV/f.u.) | (eV) | (THz) | Transition | cation | bility |
| AlCrV₃S₈ | 2.635 | 0.386 | 5.81 | SIT | MIT | 0.9545 |
| AlCrV₃Se₈ | 3.167 | 0.194 | 3.77 | SIT | MIT | 0.9472 |
| AlTaV₃Se₈ | 0.562 | 0.573 | 3.90 | MIT | MIT | 0.9822 |
| AlV₄Se₈ | 1.063 | 0.464 | 4.08 | MIT | MIT | 0.9869 |
| GaV₄Se₈ | 1.180 | 0.435 | 4.09 | MIT | MIT | 0.9869 |
| InCrV₃S₈ | 2.593 | 0.397 | 4.75 | SIT | MIT | 0.9709 |
| InCrV₃Se₈ | 3.098 | 0.216 | 5.81 | SIT | MIT | 0.9472 |
| InMo₄Se₈ | −0.693 | 0.624 | 4.55 | MIT | MIT | 0.8961 |
| InNbMo₃Se₈ | −0.660 | 0.587 | 4.44 | SIT | MIT | 0.9486 |
| InTaMo₃Se₈ | −0.878 | 0.625 | 4.25 | SIT | MIT | 0.9486 |
| InWMo₃Se₈ | −0.988 | 0.626 | 4.43 | MIT | MIT | 0.8760 |
| InWV₃S₈ | 0.090 | 0.582 | 5.83 | SIT | MIT | 0.9221 |

By focusing on the most promising materials architectures and synthesizable chemistry structures mined from literature and physics-based simulations via density functional theory (DFT), the approach as discussed will significantly reduce the number of high-fidelity design evaluations from millions to hundreds and transform the rare-event discoveries to persistent innovations. As the approach is broadly applicable to materials innovation beyond MITs materials and applications that involve expensive simulations and mixed-variable inputs, the research will impact the advancement of other energy relevant technologies and address grand challenge problems, in addition to materials design.

The major deliverables include extracted synthesis procedures for related materials, new predicted MITs compounds, improved understanding of MITs microscopic mechanisms with data repository of high-quality DFT energetics and electronic structures, new ML techniques for virtual screening using NLP, new conceptual exploration and feature extraction methods using conditional variational autoencoder (CVAE), active learning, novel high dimensional LVGP techniques with multiple responses, multi-criteria reinforcement learning in Bayesian optimization, code disseminations and software development of developed methods, and market research results.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications, and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

1. A system for performing machine learning (ML) enhanced conceptual design of compound materials, comprising:
a computing device comprising at least one processor and a storage device storing computer executable code, wherein the computer executable code, when executed at the at least one processor, comprises:
a data repository, configured to store, for a specific class of compound materials, information of existing and newly discovered compounds;
a virtual screening (VS) module, configured to identify and obtain, from literatures of a knowledge base, extracted information related to key material descriptors, relevant materials, and associated synthesis procedures of the specific class of compound materials;
a ML-assisted conceptual exploration (CE) module, configured to identify candidate material families for the specific class of compound materials based on the extracted information via a combination of ML models, and to generate exogenous models of objective functions f(x, y) and constraint functions g(x, y), wherein x represents quantitative variables and y represents qualitative variables related to structures and synthesis parameters of the specific class of compound materials; and
an adaptive discovery (AD) engine, configured to generate and optimize design of the newly discovered compound materials, and to add the information of the newly discovered compound materials to the data repository, wherein the AD engine comprises:
a mixed-variable ML module, configured to perform mixed variable ML on the objective functions f(x, y) and constraint functions g(x, y) using a latent variable Gaussian process (LVGP) model;
a mixed-integer optimization (MIO) module, configured to select new samples of (x, y) combinations in Bayesian optimization (BO) using the LVGP model and the objective functions f(x, y) and constraint functions g(x, y) for mixed-integer nonlinear programming (MINLP); and
a high-fidelity evaluation (HFE) module, configured to perform density functional theory (DFT) simulation based on the candidate material families and the associated synthesis procedures;
wherein the HFE module, the mixed-variable ML module and the MIO module of the AD engine are iteratively and sequentially executed.

2. The system of claim 1, wherein the VS module is a natural language processing (NLP) based VS module, comprising:
a data retrieval module configured to download the literatures using an Application Programming Interface (API) and retrieve content texts from the literatures; and
a text mining module, configured to perform text mining on the content texts using an unsupervised probabilistic model to obtain the extracted information.

3. The system of claim 2, wherein the literatures comprise journal articles and patents.

4. The system of claim 2, wherein the text mining module comprises:
a paragraph classifier configured to performing paragraph classifying on the content texts to identify paragraphs of interest;
a token classifier configured to tokenize words of interest within the paragraphs of interest and label the tokenized words to identify the relevant materials as recognized entities; and
a recipe mapper module, configured perform entity linking to map the recognized entities to relevant information of the knowledge base, and to establish connections between entities.

5. The system of claim 1, wherein the ML models of the ML-assisted CE module comprise design of experiments (DoE) based active learning, nonlinear regression and classification, and conditional variational autoencoders (CVAEs).

6. The system of claim 1, wherein the specific class of compound materials is a metal-insulation transitions (MITs) compound.

7. The system of claim 6, wherein the relevant materials comprise:
known MITs compounds;
unidentified potential MITs compounds with shared similarities; and
non-MITs materials.

8. The system of claim 6, wherein the ML-assisted CE module is configured to:
receive the extracted information from the VS module as input;
perform initial DoE and CVAE deep learning feature extraction to capture all known MITs and non-MITs compounds of the specific class of compound materials for subsequent model training and validation;
construct a classification model using existing dataset of compositions and structures of MITs and relevant non-MITs compounds extracted, raw candidate MIT materials and possible synthesis parameters, latent space representation of existing MITs materials, frequently used keywords in existing papers on MITs materials, all existing MITs materials, and relevant non-MITs materials, and predict, using the classification model, the candidate material families;
perform active learning of responses with regression models;
perform CVAE deep learning for generating synthesis recipes; and
obtain exogenous regression models for cost and performance.

9. The system of claim 1, wherein in the mixed variable ML module, the LVGP model performs latent variable mapping to transform the qualitative variables y into latent variables z in a two dimensional (2D) latent space to achieve physics-based dimension reduction.

10. The system of claim 1, wherein the quantitative variables x comprise:
operating pressure of a material;
stress of the material;
temperature of the material;
carrier density of the material;
fractional site occupancy of the material;
synthesis time;
synthesis temperature;
synthesis pressure; and
synthesis pH value.

11. The system of claim 1, wherein the qualitative variables y comprise:
architecture of a material;
stoichiometry of the material;
composition of the material;
type of reaction; and
processing procedure.

12. A method for performing machine learning (ML) enhanced conceptual design of compound materials, comprising:
providing a knowledge base with literatures related to a specific class of compound materials;
performing virtual screening (VS) using a VS module to identify and obtain, from the literatures of the knowledge base, extracted information related to key material descriptors, relevant materials, and associated synthesis procedures of the specific class of compound materials;
performing ML-assisted conceptual exploration (CE) using a CE module to identify candidate material families for the specific class of compound materials based on the extracted information via a combination of ML models, and to generate exogenous models of objective functions f(x, y) and constraint functions g(x, y), wherein x represents quantitative variables and y represents qualitative variables related to structures and synthesis parameters of the specific class of compound materials; and
performing adaptive discovery (AD) using an AD engine to generate and optimize design of the newly discovered compound materials, and to add the information of the newly discovered compound materials to a data repository,
wherein the AD engine comprises:
a mixed-variable ML module, configured to perform mixed variable ML on the objective functions f(x, y) and constraint functions g(x, y) using a latent variable Gaussian process (LVGP) model;
a mixed-integer optimization (MIO) module, configured to select new samples of (x, y) combinations in Bayesian optimization (BO) using the LVGP model and the objective functions f(x, y) and constraint functions g(x, y) for mixed-integer nonlinear programming (MINLP); and
a high-fidelity evaluation (HFE) module, configured to perform density functional theory (DFT) simulation based on the candidate material families and the associated synthesis procedures;
wherein the HFE module, the mixed-variable ML module and the MIO module of the AD engine are iteratively and sequentially executed.

13. The method of claim 12, wherein the VS module is a natural language processing (NLP) based VS module, comprising:
a data retrieval module configured to download the literatures using an Application Programming Interface (API) and retrieve content texts from the literatures; and
a text mining module, configured to perform text mining on the content texts using an unsupervised probabilistic model to obtain the extracted information.

14. The method of claim 13, wherein the text mining module comprises:
a paragraph classifier configured to performing paragraph classifying on the content texts to identify paragraphs of interest;
a token classifier configured to tokenize words of interest within the paragraphs of interest and label the tokenized words to identify the relevant materials as recognized entities; and
a recipe mapper module, configured perform entity linking to map the recognized entities to relevant information of the knowledge base, and to establish connections between entities.

15. The method of claim 12, wherein the ML models comprise design of experiments (DoE) based active learning, nonlinear regression and classification, and conditional variational autoencoders (CVAEs).

16. The method of claim 12, wherein the specific class of compound materials is a metal-insulation transitions (MITs) compound.

17. The method of claim 16, wherein the relevant materials comprise:
known MITs compounds;
unidentified potential MITs compounds with shared similarities; and
non-MITs materials.

18. The method of claim 16, wherein the ML-assisted CE further comprises:
receiving the extracted information from the VS module as input;
performing initial DoE and CVAE deep learning feature extraction to capture all known MITs and non-MITs compounds of the specific class of compound materials for subsequent model training and validation;
constructing a classification model using existing dataset of compositions and structures of MITs and relevant non-MITs compounds extracted, raw candidate MIT materials and possible synthesis parameters, latent space representation of existing MITs materials, frequently used keywords in existing papers on MITs materials, all existing MITs materials, and relevant non-MITs materials, and predicting, using the classification model, the candidate material families;
performing active learning of responses with regression models;
performing CVAE deep learning for generating synthesis recipes;
obtaining exogenous regression models for cost and performance;

19. The method of claim 12, wherein in the mixed variable ML module, the LVGP model performs latent variable mapping to transform the qualitative variables y into latent variables z in a two dimensional (2D) latent space to achieve physics-based dimension reduction.

20. The method of claim 12, wherein the quantitative variables x comprise:
operating pressure of a material;
stress of the material;
temperature of the material;
carrier density of the material;
fractional site occupancy of the material;
synthesis time;
synthesis temperature;
synthesis pressure; and
synthesis pH value.

21. The method of claim 12, wherein the qualitative variables y comprise:
architecture of a material;
stoichiometry of the material;
composition of the material;
type of reaction; and
processing procedure.

22. A non-transitory tangible computer-readable medium storing computer executable instructions which, when executed by one or more processors, cause the method of claim 12 to be performed.
